(19) United States
(12) Patent Application Publication
(10) Pub. No.: US 2019/0167523 A1
(43) Pub. Date: Jun. 6, 2019

(54) MEDICAMENT DISPENSER

(71) Applicant: INFIELD MEDICAL, LLC, Morrisville, NC (US)

(72) Inventors: Edward P. Browka, Morrisville, NC (US); Amanda H. Schaffers, Morrisville, NC (US); David L. Foshee, Morrisville, NC (US); Eli B. Nichols, Morrisville, NC (US); David Orenstein, Morrisville, NC (US); Theodore J. Mosler, Morrisville, NC (US)

(21) Appl. No.: 16/323,461

(22) PCT Filed: Aug. 4, 2017
(86) PCT No.: PCT/US17/45478

§ 371 Date: Feb. 5, 2019

Related U.S. Application Data
(60) Provisional application No. 62/371,551, filed on Aug. 5, 2016.

(51) Int. Cl. A61J 1/20; A61J 7/00

(52) U.S. Cl. CPC A61J 1/2037; A61F 9/0026; A61J 7/0053; A61J 1/201

(57) ABSTRACT

A dispensing device, comprising: a piston-cylinder assembly, the assembly comprising an outlet in fluid communication with a one-way valve and an inlet in fluidic communication with a valve assembly; the one-way valve configured to dispense a liquid from a medicament container, the valve assembly fluidically coupled to the one-way valve, the valve assembly having at least one cannula and at least one flow control member; the piston configured to draw an amount of fluid through the valve assembly and inlet into the cylinder, and to expel the amount of liquid through the one-way valve; and an actuator assembly coupled to and biasing the piston with stored energy.

### TECHNICAL FIELD

This disclosure generally relates to a medicament dispenser comprising a piston-cylinder assembly, a valve assembly, and actuator assembly configured to receive and dispense a source of medicament.

### BACKGROUND

Conventionally drops are applied to the eye, mouth or other mucus membranes with a dropper generally by squeezing a plastic container fitted with a tapered nozzle. This manner of application has a number of disadvantages such as the requirement to align the tapered nozzle precisely with the eye as well as from anticipation of the drop and involuntary blinking, both of which may result in the drop of medicament not being delivered to the ocular space. Furthermore, and particularly applicable to self-administration of eye drops, is the problem of uncertain dosage originally applied and the indeterminate fraction thereof that is squeezed out of the eye by reflex blinking.

Continued use of eye drops in certain instances is required for medical treatment. Thus, for example, in the treatment of glaucoma it is conventional to require the administration to the ocular space a number of times per day for life. The necessity of continued usage of eye drops as in the foregoing instance usually requires the drops to be self-administered and the present disclosure is particularly adapted to improve this application.

### SUMMARY

In a first embodiment, a valve assembly comprising a piston-cylinder assembly configured to receive and dispense a liquid from a container, the piston-cylinder assembly having at least one fluid inlet, at least one vent inlet, and at least one fluid outlet; a cannula in fluidic communication with the vent inlet; a sheath surrounding the cannula and providing a fluid path between the cannula and the sheath, the sheath in fluid communication with the fluid inlet and the fluid outlet; an actuator assembly providing stored energy and release of the stored energy, the actuator assembly coupled to the piston; and a container manipulation system configured to align and present a container with the cannula and the sheath.

In a second embodiment, a dispensing device is provided, the dispensing device comprising: a piston-cylinder assembly, a fluidic system fluidically coupled to the piston-cylinder assembly, the fluidic system comprising a valve assembly, the valve assembly comprising: a cannula having a piercing distal end; a proximal end separated by a length; and at least one side port in proximity to the piercing distal end; and the at least one side port fluidically coupled to a vent; a one-way valve fluidically coupled to the vent; a sheath surrounding the length of cannula, the sheath having a distal end; a proximal end; at least one side opening; and a fluid path between the at least one side opening and a portion of the length of the cannula; a one-way valve fluidically coupled to the flow path; an outlet in fluid communication with the cylinder and the flow path; the piston configured to draw an amount of liquid through the valve assembly into the cylinder, and to expel the amount of liquid; and an actuator assembly coupled to and biasing the piston with stored energy.

In a third embodiment, a dispensing device is provided, the dispensing device comprising: a housing comprising: a piston-cylinder assembly; a fluidic system fluidically coupled to the piston-cylinder assembly; a container manipulation assembly for receiving and fluidically coupling contents of a container to the fluidic system; the fluidic system comprising a valve assembly, the valve assembly comprising: a cannula having a piercing distal end; a proximal end separated by a length; and at least one side port in proximity to the piercing distal end; and the at least one side port fluidically coupled to a vent; a one-way valve fluidically coupled to the vent; a sheath surrounding the length of cannula, the sheath having a distal end; a proximal end; at least one side opening; and a fluid path between the at least one side opening and a portion of the length of the cannula; a one-way valve fluidically coupled to the flow path; an outlet in fluid communication with the cylinder and the flow path; the piston configured to draw an amount of liquid through the valve assembly into the cylinder, and to expel the amount of liquid; the container manipulation assembly configured for arranging the cannula and the sheath to access contents of the container; and an actuator assembly coupled to and biasing the piston with stored energy.

In a third aspect, alone or in combination with any of the previous aspects of the first embodiment, the actuator operably coupled to a stored energy source.

In a fourth aspect, alone or in combination with any of the previous aspects of the first embodiment, the actuator assembly is configured with a first configuration and a second configuration, the first configuration capable of urging the piston in a first direction within the cylinder and activating a stored energy source; the second configuration capable of releasing the stored energy activated by the first configuration and urging the piston in a second direction within the cylinder opposite the first direction.

In order to understand the invention and to see how it may be carried out in practice, embodiments will now be described, by way of non-limiting example only, with reference to the accompanying drawings, in which:

### BRIEF DESCRIPTION OF THE DRAWINGS

FIG. 1A and FIG. 1B depict perspective views of an embodiment of the present disclosure.

FIG. 2 depicts an exploded view of the device of FIG. 1A.

FIG. 3 depicts an exploded view of a fluid flow system of the present disclosure.

FIG. 4A and FIG. 4B depict a plan and side view, respectively, of the device of FIG. 1A.

FIG. 5 depicts a sectional view of FIG. 4B along section line 5-5.

FIG. 6A and FIG. 6B depict a plan and side view, respectively, of the device of FIG. 1A with a medicament container.

FIG. 7 depicts a sectional view of FIG. 6B along section line 7-7.

FIG. 8A and FIG. 8B depict a plan and side view, respectively, of the device of FIG. 1B.

FIG. 9 depicts a sectional view of FIG. 8B along section line 9-9.

FIG. 10 depicts a sectional view of FIG. 4A along section line 10-10.

FIG. 11 depicts a sectional view of FIG. 6A along section line 11-11.

FIG. 12 depicts a sectional view of FIG. 8A along section line 12-12 in a primed, activated state.

FIG. 13 depicts a sectional view of FIG. 8A along section line 12-12 during activation.

FIG. 14 depicts a sectional view of FIG. 8A along section line 12-12 with an alternate activation embodiment.

FIG. 15A and FIG. 15B depict perspective views of an embodiment of the present disclosure.

FIG. 16A depicts an exploded view of the device of FIG. 15A.

FIG. 16B depicts a partial exploded view of the activation mechanism of the present disclosure.

FIG. 17 depicts an exploded view of a fluid flow system of the present disclosure.

FIG. 18A, FIG. 18B, FIG. 18C, and FIG. 18D depict a first perspective view, a second perspective view rotated about 90 degrees from the first perspective view, a top view, and a bottom view, respectively, of a container manipulation system of the present disclosure.

FIG. 19A depicts a side view of the device of FIG. 15A.

FIG. 19B depicts a sectional view along section line 19A-19A of the device of FIG. 19A without a medicament container.

FIG. 20A depicts a top plan view of the device of FIG. 15A.

FIG. 20B is a side view of the device of FIG. 15A.

FIG. 21 is a sectional view of FIG. 20B along section line 21-21.

FIG. 22 is a sectional view of FIG. 20A along section line 22-22.

FIG. 23 is a sectional view of FIG. 20A along section line 23-23.

FIG. 24 is a sectional view of FIG. 20A along section line 24-24.

FIG. 25A is a top plan view of FIG. 15B.

FIG. 25B is a side view of FIG. 15B.

FIG. 26 is a sectional view of FIG. 25B along section line 26-26.

FIG. 27 is a sectional view of FIG. 25A along section line 27-27.

FIG. 28 is a sectional view of FIG. 27 along section line 28-28.

FIG. 29A is a top plan view of the device of FIG. 15B in an activated state.

FIG. 29B is a side view of the device of FIG. 15B in an activated state.

FIG. 30 is a sectional view of FIG. 29B along section line 30-30.

FIG. 31 is a sectional view of FIG. 29A along section line 31-31.

FIG. 32 is a partial sectional view of section 32 of FIG. 29A.

FIG. 33 is a sectional view of FIG. 29A along section line 33-33.

FIG. 34 is a sectional view of FIG. 29A along section line 34-34.

FIG. 35A is a top plan view of the device of FIG. 15B in a final state.

FIG. 35B is a side view of the device of FIG. 15B in a final state.

FIG. 36 is a sectional view of FIG. 35B along section line 36-36.

FIG. 37 is a sectional view of FIG. 35A along section line 37-37.

FIG. 38 is a sectional view of FIG. 35A along section line 38-38.

FIG. 39 is a sectional view of FIG. 35A along section line 39-39.

FIG. 40 is a side view of the device of FIG. 15B in an inverted state.

FIG. 41 is a sectional view of the device of FIG. 40 along section line 41-41.

FIG. 42 is a partial sectional view of section 42 of FIG. 41.

FIG. 43 is a partial cutaway view of FIG. 40.

### DETAILED DESCRIPTION

An apparatus and method for applying medicament to the eye by urging an amount of fluid from a medicament container through an inlet into a cylinder via a flow control valve. The medicament is urged with sufficient velocity from the cylinder with a piston through a one-way valve to minimize delivery loss via blinking of the eye during self-administration. In one aspect of the present disclosure, the present device is devoid of a pressurized gas source to assist in the dispensing of the medicament.

The embodiments herein are exemplarily described in the context of a device comprising a modular system in which the device and the medicament liquid source are provided separately, or at least independent of each other. A liquid medicament source can then be independently selected, and coupled to the present device as desired. This enables the same device to be used repeatedly and/or for different treatments. Such a device, if used for institutional use, can be at least partially automated or provided as a kit or combination, so as to provide for such medicament selection. It is understood that the present disclosure can be provided as a single unit, or a single use device, the medicament or its container integral with the device.

Devices disclosed herein can be for personal or hand held use, or for use on a more regular basis in healthcare settings. For whatever use, the device is configured for ensuring a proper spacing between the device and the eye to be targeted, and this can be adjusted, particularly in the devices adapted for institutional use. In this respect, it will be noted that the dispensing mechanisms disclosed and described in the present disclosure are capable of discharging a form of liquid medicament, e.g., droplets, substantially horizontally or vertically over a minimum distance so as not requiring a user or healthcare provider to operate the device directly above an eye, e.g., not requiring solely the use of gravity to target drops of medicament.

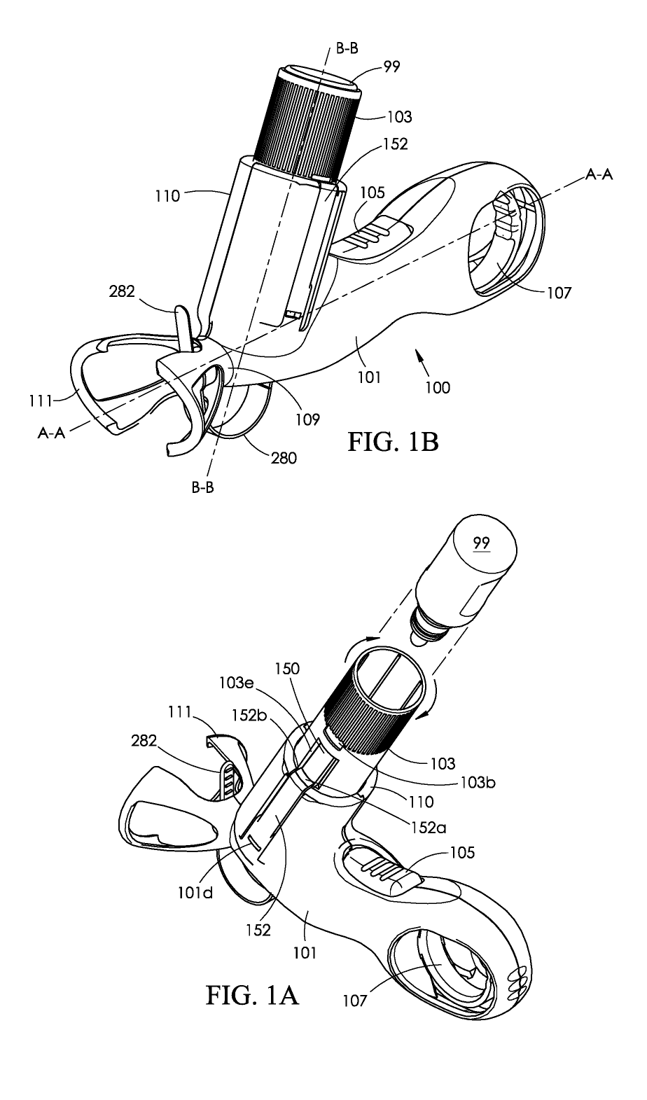

With reference to FIGS. 1A and 1B, dispenser 100 is shown in perspective views comprising a carriage receiving member 110 presenting an opening for receiving a carriage manipulation system comprising a carriage member 103, the carriage member 103 configured for receiving medicament container 99, the carriage receiving member 110 integral with housing 101. Device 100 further comprises optional projection 112. Projection 112 is configured for assisted locating and/or positioning the dispensing device 100 in proximity to a target location, for example, an eye or mucous membrane. Projection 112 can be configured in a variety of sizes and may be readily removed at collar 109 and attached to device 100.

Carriage member 103 is shown engaged with carriage receiving member 110 that is integral with dispenser 100. In one aspect, device 100 can be provided with carriage member 103 separate and apart from carriage receiving member 110. In one aspect, as shown in FIGS. 1A and 1B, carriage receiving member 110 projects from the longitudinal axis A-A of device 100. In one aspect, as shown, carriage receiving member 100 projects at an angle less than 90° from the longitudinal axis A-A of device 100. In other aspects, carriage receiving member 110 can project essentially perpendicular to the longitudinal axis A-A of device. Button 105 is operably coupled to an actuating system 111 internal to the housing 101.

With reference to FIG. 1A, carriage member 103 is shown receiving medicament container 99. With reference to FIG. 1B, upon receipt of the medicament container 99 by carriage member 103, the carriage member is configured for engaging with the carriage receiving member 110, for example, with corresponding external and internal threading.

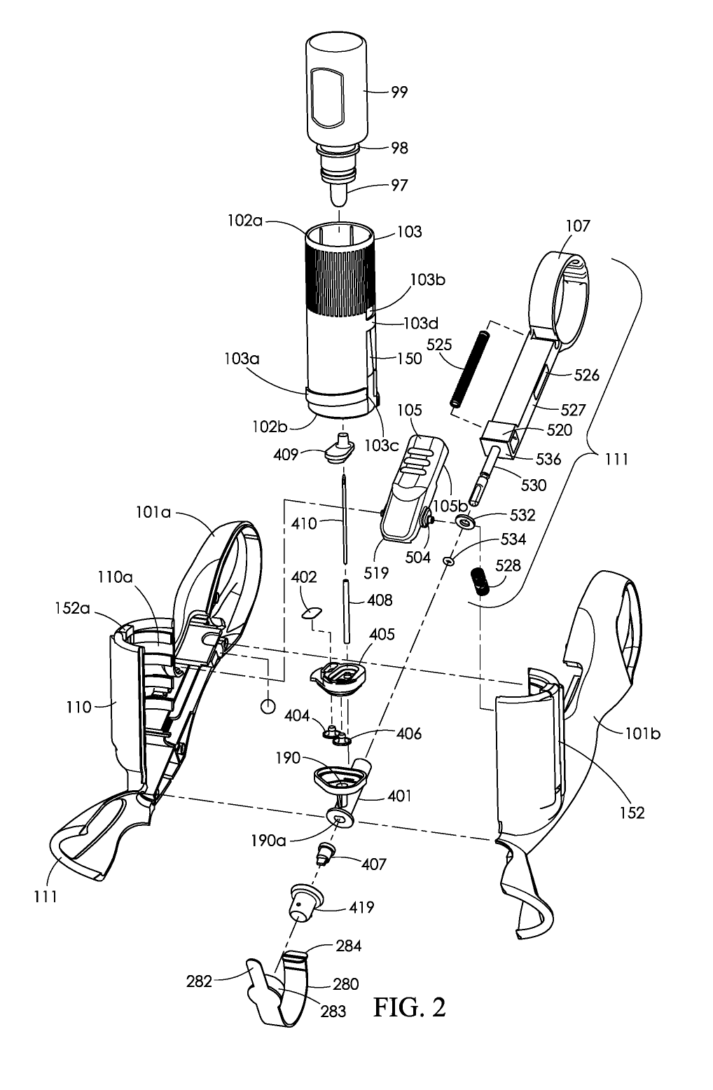

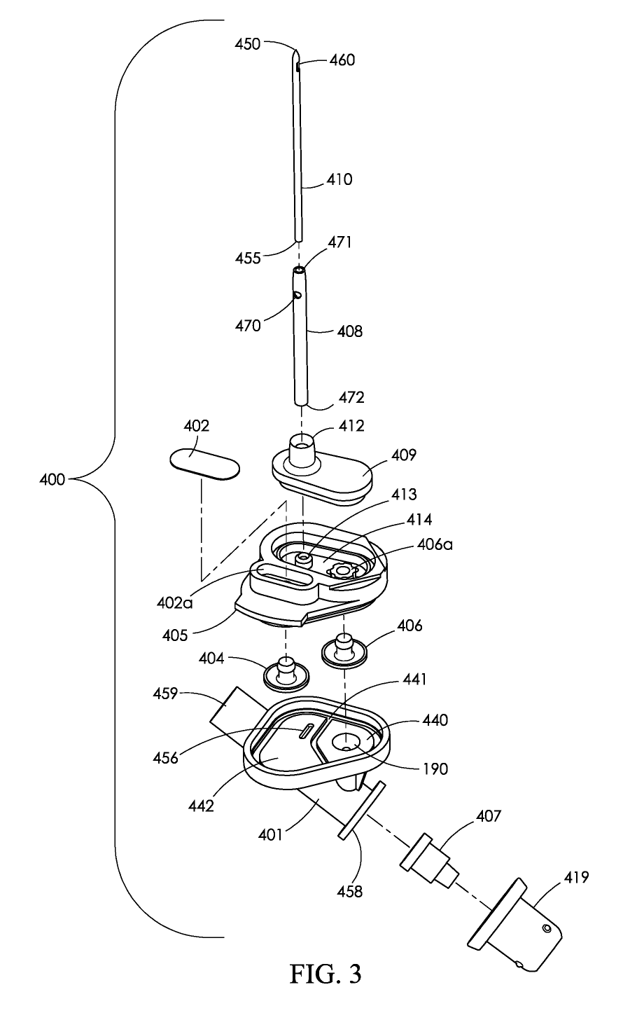

With reference to FIGS. 2 and 3, an exploded view of the device of FIG. 1A (without medicament container 99) is provided, showing carriage member 103 received by carriage receiving member 110, and that it at least partially surrounds a portion of a cannula 410 having sheath 408.

In one aspect, cannula 410 is configured to be essentially centered in carriage receiving member 110. In one aspect, cannula 410 is configured to be essentially centered in carriage receiving member 110 and carriage member 103.

Valve assembly 400 positioned between housing components 101a and 101b, fluidically couples a fluidic system comprising valve assembly 400 to piston-cylinder components of an actuating assembly 333. Valve assembly 400 comprises an upper valve housing 405 being coupled on one (lower) side to a lower valve housing 401 and a flange seal 409 on the other (upper) side.

With reference to FIG. 3, flange seal 409 comprises seat 412 for receiving proximal end 455 of cannula 410 and proximal end 472 of sheath 408. Flange seal 409 couples with upper valve housing 405 to provide a fluid reservoir 414. Fluid reservoir 414 contains coupling 406a and ferrule 413 spatially separated from coupling 406a. Upper valve housing 405 comprises an isolated filter compartment 402a and filter 402 adjacent the flange seal 409.

Upper valve housing 405 is couplable to lower valve housing 401 and together with partition 441 provides a fluid compartment 440 isolated from vent compartment 442 in between the respective valve housings, each respective compartment having associated there with flow control valves 406 and 404, respectively. Coupling 406a secures flow control valve 406 in-between upper and lower valve housing components. Flow control valve 406 fluidically couples fluid reservoir 414 and fluid compartment 440 with sheath 408 and the at least one side opening 470. Flow control valve 404 fluidically couples vent compartment 442 and vent filter housing 402a with cannula 410 and the at least one side-port 460. In this configuration, the venting of device 100 is essentially isolated from the liquid flow path.

Lower housing 405 comprises opposing flange ends 458, 459. Flange 458 couples with flange cap 419 and encloses a one-way dispensing valve 407. Opposite flange 459 couples flow path 190 with actuator system 333 discussed below.

With specific reference to the expanded exploded view of FIG. 3, fluidic cannula 410 has a pointed distal end 450, at least one side-port 460, and a proximal end 455. Proximal end 455 of cannula 410 is received by a sheath 408 having a distal opening 471 and a proximal opening 472. Sheath 408 comprises at least one side opening 470 positioned between is distal opening 471 and proximal opening 472. In one aspect, the outer diameter of cannula 410 is configured such that it is smaller than the inner diameter of sheath 408 such that fluid may freely flow between the outer diameter of the cannula and the inner diameter of the sheath. In some aspects, the spacing between the inner diameter of the sheath and the outer diameter of the cannula can be between about 0.0005 inches (about 0.00127 centimeter) to about 0.005 inches (about 0.0127 centimeter). Other spacing of the inner diameter of the sheath and the outer diameter of the cannula can be used. The inner diameter of sheath 408 from its distal opening 471 and proximal opening 472, surrounds cannula 410 from its proximal end 455 up to but not including or otherwise obfuscating or blocking the one or more side-port 460 or the pointed distal end 450. In other words, the at least one side-port 460 is spatially (e.g., vertically from the housing, with reference to axis B-B of FIG. 1B) separated from the at least one side opening 470 of sheath 408. The spatial separation of the at least one side-port 460 and the at least one side opening 470 can be determined, for example, based on the physical parameters of the medicament container and other physical parameters of the device, as further discussed below.

In one aspect, distal opening 471 is reduced in inner diameter so as to secure and/or seal outer diameter of cannula 410 with sheath 408 and thus direct liquid flow essentially through the at least one side opening 470. Securing distal opening 471 with cannula can be by press-fit, welding, adhesive, sonic, and the like. In one aspect, there can be no intentional sealing of opening 471 and the cannula. In this configuration, side-port 460 of cannula 410 functions efficiently as an air vent while the at least one side opening 470 of sheath 408 functions to receive liquid from the medicament container 99 about the inner diameter of the sheath and allows the liquid to flow around the outer diameter of cannula 410 into fluid reservoir 414. In one aspect, liquid flow between the at least one side-port 460 and proximal end 455 of cannula 410 is prevented. In another aspect, liquid flow between the at least one side-port 460 and proximal end 455 of cannula 410 is prevented and liquid flow is restricted to the space between sheath 408 cannula 410. In one aspect, the side-port 460 is arranged to be about 180 degrees rotated from the side opening 470 to maximize fluid draw and venting exchange. In other aspects, using longitudinal axis B-B as reference, the side-port 460 is positioned such that it is vertically above the side opening 470, relative to the housing. In one aspect, the side-port 460 is arranged to be about 180 degrees rotated from the side opening 470, and, using longitudinal axis B-B as reference, the side-port 460 is positioned such that it is vertically above the side opening 470, relative to the housing.

With specific reference to the expanded exploded view of FIG. 3, proximal end 455 of cannula within sheath 408 is received by seat 412 of flange seal 409, whereas the proximal end 455 of cannula proceeds thru seat 412 to ferrule 413 and further extends into vent compartment 442, terminating at vent seat 456 so as to provide an isolated vent path from the at least one side-port 460 of the cannula and the vent compartment 442. Vent seat 456 may be an elevated protrusion projecting from a surface of vent compartment 442 or may be a depression in the surface of the vent compartment, and maybe of a size and shape so as not to completely obstruct the distal end 450 of the cannula 410 and to allow for venting.

Proximal opening 472 of sheath 408 seats in seat 412 of the flange seal 409 so as to provide an isolated fluid path to fluid reservoir 414. In one aspect, outer diameter of sheath 408 is secured to seat 412 with an adhesive. Seat 412 is designed to receive proximal opening 472 of sheath 408 so as to avoid contamination of the fluid path (the inner diameter of sheath 408) by the adhesive. Tapering and other design features can also be used to provide a liquid tight seal between the outer diameter of sheath 408 and seat 412.

With reference back to exploded view FIG. 2, actuating system 333 is shown fluidically coupled to flow channel 190 of flow path 190a and flange end 459 of lower valve housing 401, comprises a piston 530 sealably engaged to the fluid flow path 190a of lower valve housing 401. Piston 530 and fluid flow path 190a provide a piston-cylinder arrangement. In one aspect, a portion of fluid flow path 190a provides a cylinder 190b within lower valve housing 401 for receiving piston 530 and allowing for reversible translation of the piston within the cylinder essentially parallel with the longitudinal axis A-A and essentially opposed to longitudinal axis B-B. O-ring 534 provides a liquid-tight seal of piston 530 in cylinder 190b. Gasket 532 provides adjustment of piston stroke length and medicament dosing/dispensing amount and can also provide a reduction or elimination of sound and/or vibration. Piston 530 is attached or otherwise integral at its opposing end to member 527. Member 527 is generally an elongated structure of two parallel projecting surfaces providing a opening there between terminating at one end, as shown, in an annular ring configured for pulling, in a generally lateral direction parallel to the longitudinal axis A-A of device 100, for example, by a digit of a human hand. Stored energy 525, exemplified as a spring, is configured within opening between parallel projecting surfaces of member 527. Upper surface 520 is raised from the parallel projecting surfaces of member 527 and terminates in a lip 536 proximal to piston 530.

Member 527 and actuating button 105 are configured to be positioned within the housing 101. Actuating button 105 comprises pivot points pivotally configured in housing 101. Biasing means 528 are positioned at end 105b of button 105 so as to allow the button to reversibly operate in a direction essentially perpendicular to the longitudinal axis A-A of device 100. Button 105 comprises, at its opposite end, a projecting lip 519 that engages lip 536 of member 527 and allows for control of the stored energy 525 and the dispensing of medicament by the device 100. Projecting lip 519 can be presented as gear teeth with complementary teeth presented on upper surface 520 of member 527.

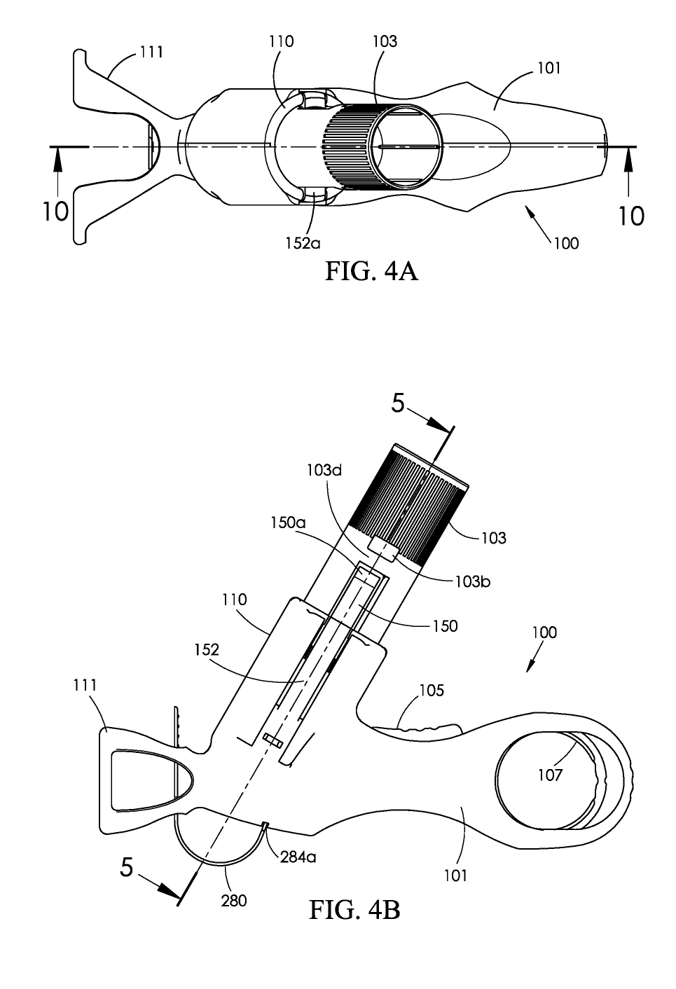

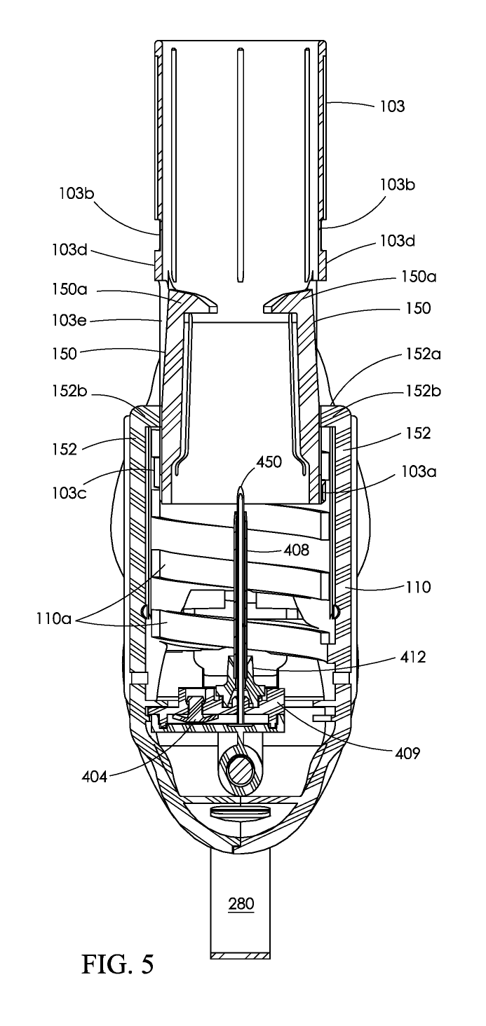

The function and structural relationships of carriage receiving member 110 and carriage member 103 are now discussed. With reference to FIGS. 1A ,1B, 4A, 4B, and FIG. 5, depicting a sectional view of FIG. 4B along section line 5-5, carriage member 103 is shown as an elongated cylinder having a first end 102a and a second end 102b that is generally centered and aligned with longitudinal axis B-B. Male threads 103a, in proximity to the second end 102b, is configured to partially encircle the outer diameter of carriage member 103 and provide edges 103c at both terminal ends of the male threads 103a. Male threads 103a is configured to be received by female threads 110a of housing 101.

With reference to perspective views FIGS. 4A, 4B, 6A, 6B, and 8A and 8B, and their corresponding sectional views of FIGS. 5, 7, and 9, along sectional lines 5-5, 7-7, and 9-9, respectively, the device 100 is depicted without medicament container 99, with medicament container 99 inserted in carriage member 103, and with medicament container and carriage member 103 fully threaded into carriage receiving member 110, respectively. The structural and functional interaction between the carriage member 103, carriage receiving member 110, and the medicament container 99, carriage member 103, are now described.

With reference to FIGS. 1A, and 5, depicting a sectional view of FIG. 4B along section line 5-5, device 100 is shown in an initial configuration, without medicament container 99. Upon assembly, at least a portion of elongated members 150 is presented in a cavity formed through carriage member 103. Elongated members 150 are approximately attached to carriage member 303 and project parallel to longitudinal axis B-B. As shown, each of the 2 two elongated members 150 terminate in a distal end corresponding to projecting protrusions 150a, shown projecting essentially horizontally. Protrusions 150a project into the interior of carriage member 103. The width of the cavity containing elongated members 150, measured along the outer perimeter of carriage member 103 are sized to receive the total width of protrusions 152a of the carriage receiving member 110. In this assembled configuration, cavity wall edge 103e, exposed by the inward inflection of elongated members 150 by elongated retaining members 152, engages with edge surfaces 152b of protrusions 152a of retaining members 152, preventing carriage member 103 from threading (either clockwise or counterclockwise) with carriage receiving member 110 in the absence of an inserted medicament container 99, as further discussed below. Upon assembly, and in this initial configuration, second end 102b and male threads 103a of carriage member 103 extends into carriage receiving member 110 to at least partially engage female threads 110a. The pre-engagement of male threads 103a and a lip formed on the interior diameter of the opening of carriage receiving member 110 prevents carriage member 103 from being completely removed, either before or after medicament container 99 is inserted.

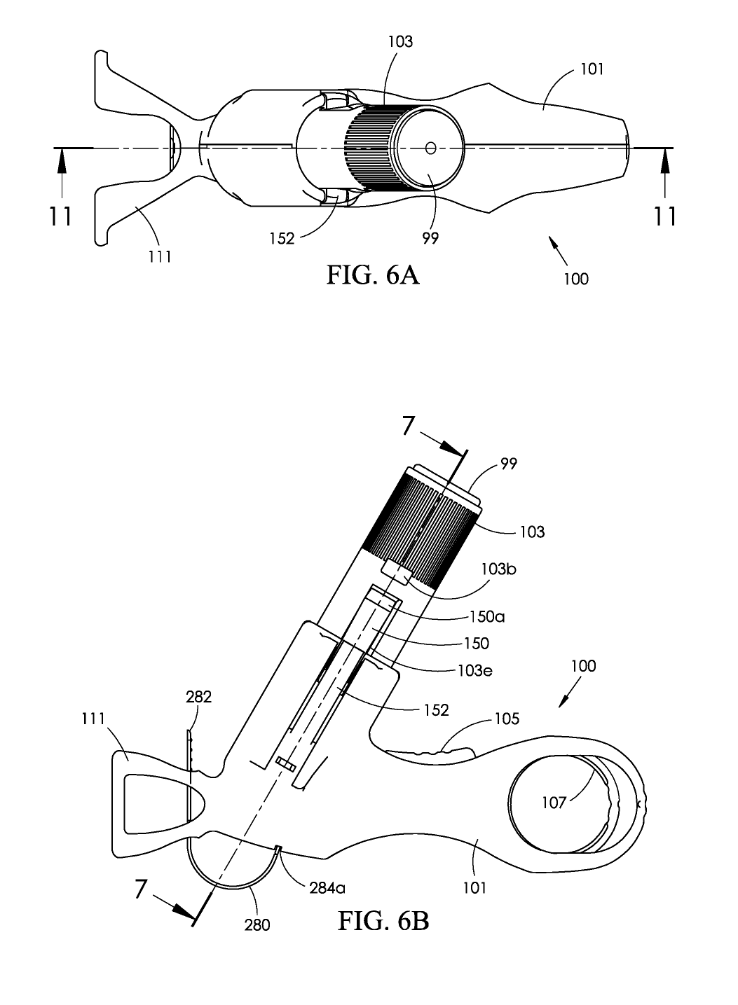

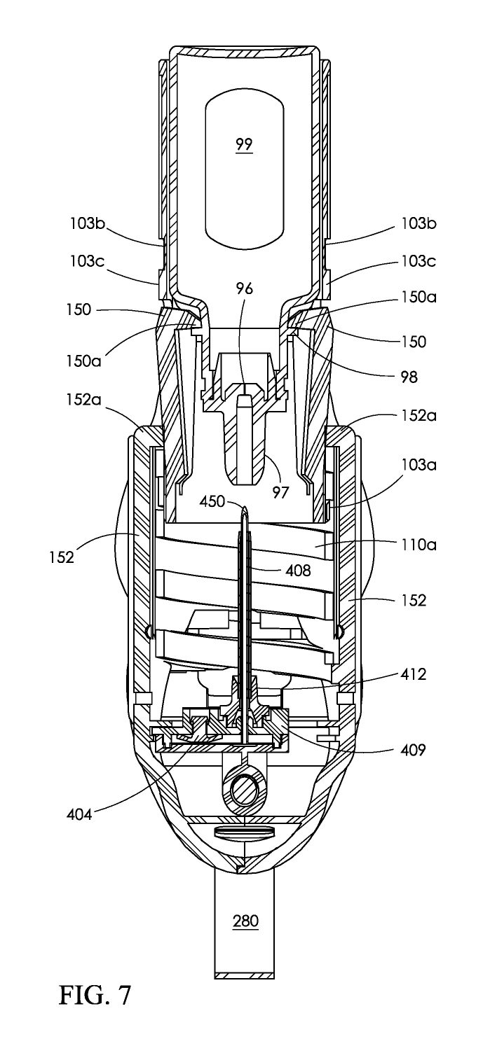

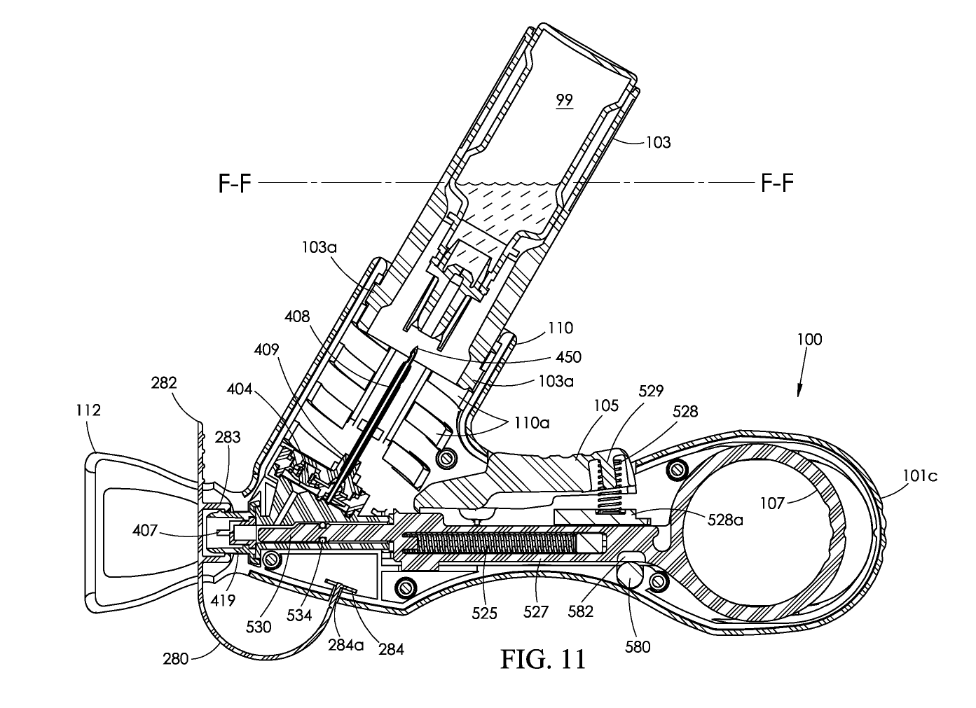

With reference now to FIGS. 7 and 11, depicting a sectional view of FIG. 6B along section line 7-7 and a sectional view of FIG. 6A along section line 11-11, respectively, device 100 is depicted in a first configuration, where a medicament container 99 has been introduced to carriage member 103. Inwardly, facing projecting protrusions 150a of elongated members 150 allow an annular collar 98 of medicament container 99 to slidably pass and slightly outwardly bias protrusions 150a and elongated members 150 along a portion of their length. After the annular collar 98 has passed the protrusions 150a. elongated members 150 partially return to their initial configuration and restrain or prohibit the medicament container 99 from being withdrawn from carriage member 103, but are no longer flush along the entire length with the outer diameter of carriage member 103, and now extends slightly past cavity wall edge 103e. The bias applied to elongated members 150 by the inserted container 99 provides for a portion of elongated member 150 to engage with a portion of inwardly projecting protrusions 152a of corresponding retaining members 152 of carriage receiving member 110 so as to allow edges 103c of cavity of carriage member to freely rotate without engaging edge surfaces 152b of projecting protrusions 152a and thus allow one way threading of carriage member 103 with carriage receiving member 110. The composition, bias and flexibility of elongated members 150 and/or elongated retaining members 152 can be adjusted for a desired amount of flexibility/bias for repeated use as well as, for example, by tapering or providing step changes in thickness along the length of the member.

Still referencing FIG. 7, device 100 provides for retaining of medicament container 99 and aligning tapered nozzle 97 in carriage member 103 and for threading the carriage member into carriage receiving member 110. Distal end 450 of cannula 410 is aligned with tapered nozzle 97 of medicament container 99 such that threading of carriage member 103 into carriage receiving member 110 introduces distal end 450 of cannula 410 into tapered nozzle 97 of medicament container 99, thus, eliminating possible deflection and/or misalignment of the cannula with a rather small diameter orifice typical of such tapered nozzles of such medicament containers.

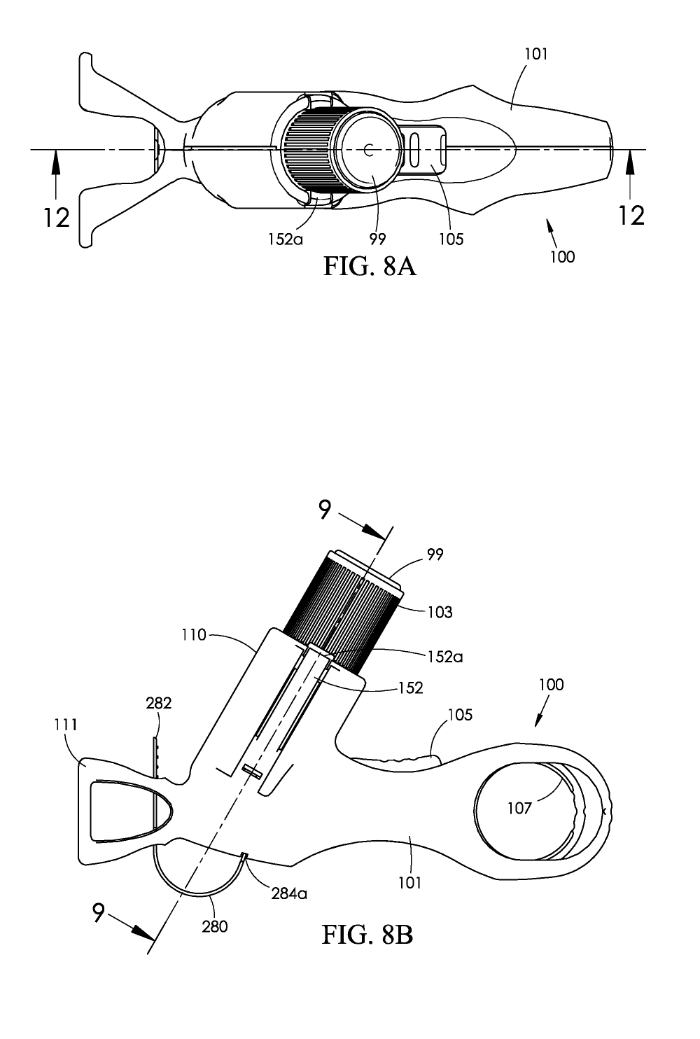

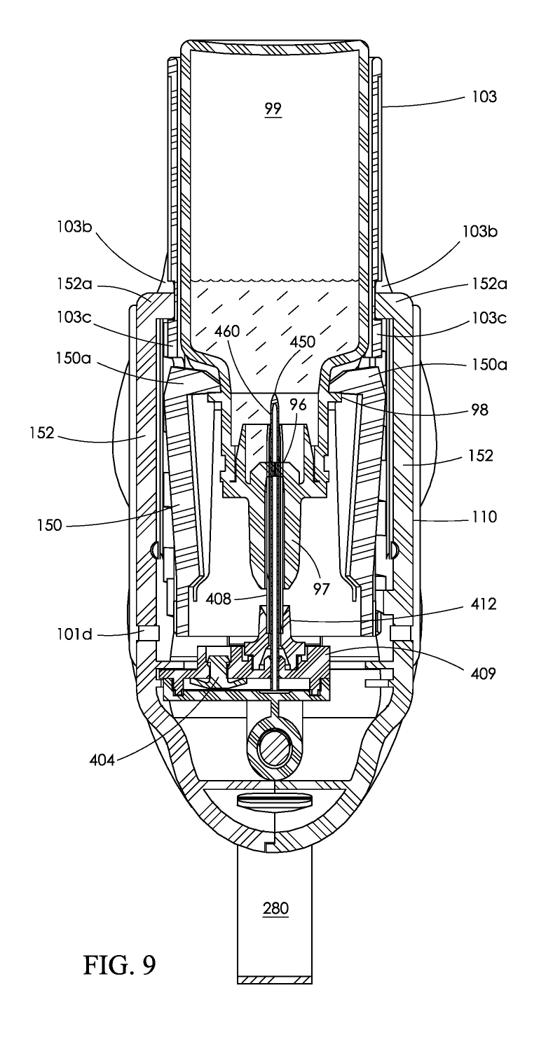

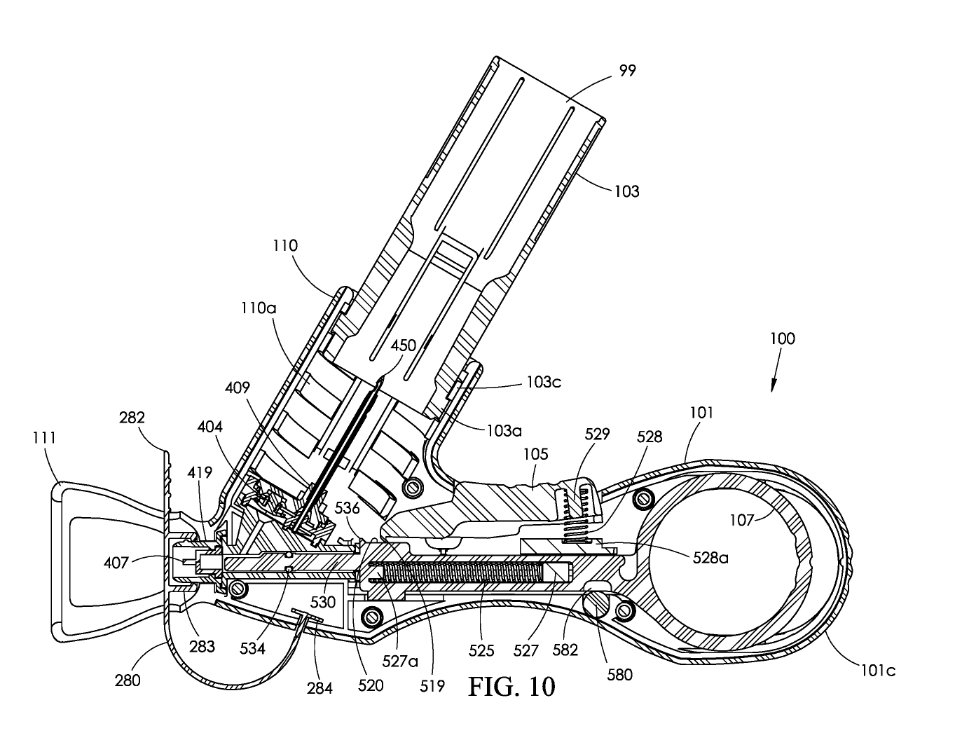

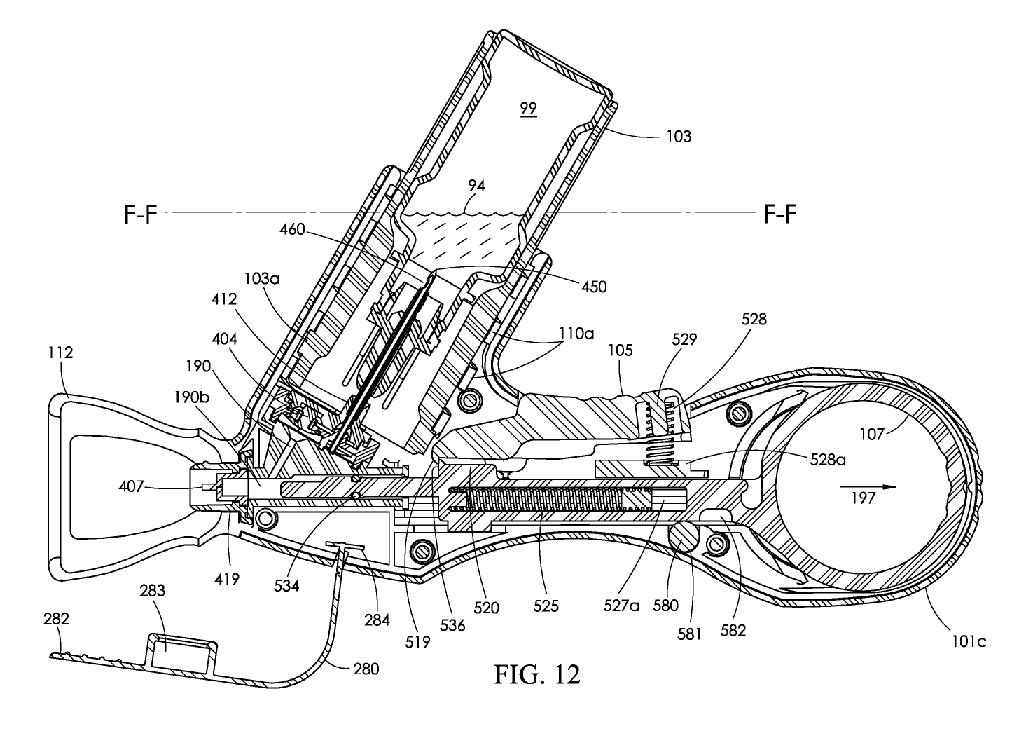

With reference now to FIGS. 9 and 12, depicting a sectional view of FIG. 8B along section line 9-9 a sectional view of FIG. 8A along section line 12-12, respectively, device 100 is presented in a second configuration with medicament container 99 and carriage member 103 fully threaded into carriage receiving member 110. In this second configuration, distal end 450 and side-port 460 of cannula 410, as well as side opening 470 of sheath 408, having been properly aligned with tapered nozzle 97, are urged through opening 96 of medicament container 99. As the opening 96 of a typical medicament container is of a small-diameter and generally constructed of a thermoplastic polymer such as polyethylene or polypropylene, in one aspect, cannula 410 is of metal or of an engineering grade plastic having higher tensile strength than that of such polyolefins. Distal end 450 of cannula 410 can be sharpened and/or beveled in a variety of ways suitable for advancing through opening 96. Likewise, distal end 471 of sheath 408 can be constructed of a rigid polymer of suitable tensile properties and/or tapered so as to engage and transverse opening 96.

Upon completion of the threading of carriage member 103 with carriage receiving member 110, protrusions 152a of the carriage receiving member are permitted to engage openings 103b adjacent section 103d of the carriage member and thus effectively reduce or prevent the reverse threading of the carriage member. In addition, projections 150a of elongated members 150 of carriage member 103 maintain the securement of medicament container 99 and prevent or eliminate its removal while providing one or more uses of the device 100. Window 101d provides for an indication of complete threading of carriage member 103 with carriage receiving member 110, for example, where male threads 103a and/or a portion of the carriage member are brightly or fluorescently colored.

In one aspect, device 100 is designed for dedicated use with a single medicament container 99 and/or whereas forcing release of either the carriage member 103 from the carriage receiving member 110 or the medicament container 99 from the carriage member 103 would cause damage, including non-operability, of the device. Device 100 and one medicament container 99 can be collectively provided as a kit. Alternatively, device 100 can be configured such that projections 150a and 152a can accept one or more special tools designed to release carriage member 103 from protrusions 152a so as to permit reverse threading of carriage member 103 as well as allowing release the medicament container 99 from protrusions 150a, so that a medicament regimen using different medicament containers can be employed. Device 100 and two or more medicament containers 99 can be collectively provided as a kit.

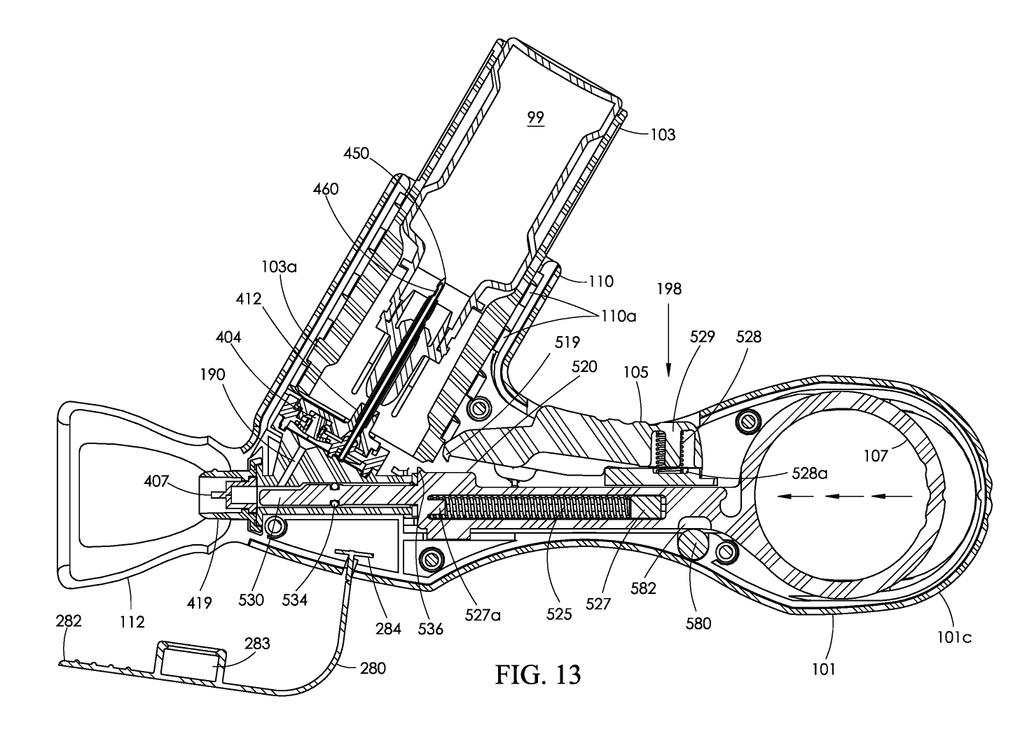

With the side-port 460 of cannula 410 and side opening 470 of sheath 408 introduced into medicament container 99, the operation of device 100 can now be described with reference to FIGS. 12-13. In this activated configuration, device 100 is configured such that the actuating system 333 can be engaged by the user and prepare device 100 for the dispensing of an amount of medicament from medicament container 99.

Thus, referencing FIGS. 12-13, that depict a sectional view of FIG. 8A along section line 12-12 in a primed, activated state; and a sectional view of FIG. 8A along section line 12-12 during activation, respectively, the use of device 100 can now be described.

FIG. 12 shows piston 530 and will ring 534 slidably received in cylinder 190b. Flow channel 190 of lower valve housing 401 is fluidically coupled to cylinder 190b and fluid compartment 440 via first flow control valve 404 that is fluidically coupled to fluid reservoir 414 that is fluidically coupled to side opening 470 of sheath 408.

First and second flow control valves 404, 406 can be check valves arranged together in opposite flow control arrangement such that liquid medicament from container 99 is restricted to flowing in one direction through fluid reservoir 414 and fluid compartment 440 by flow control valve 406 into cylinder 190b, and air is restricted to flow in the opposite direction from filter 402, vent compartment 442, flow control member 404, through cannula 410 and side-port 460. Distal end 105b of user actuating button 105 is pivotally coupled to lip 519 for engagement with lip 536 of surface 520. Stored energy 525 is positioned in space 526 of member 527 and is secured with member stop 527a.

In preparation for use, ring member 107 is urged rearward towards distal end 101c of housing 101 by a user or healthcare provider, as shown by directional arrow 197, causing stored energy 525 to compress against member stop 527a, drawing piston 530 in a first direction opposite that of the direction the medicament is to be dispensed and generally parallel to the longitudinal axis A-A, while drawing an amount of fluid from medicament container 99 into side opening 470 of sheath 480, into fluid reservoir 414 of upper valve housing 405. Flow control valve 406 is thus caused to open and release the fluid from the fluid reservoir 414 into flow channel 190 of fluid compartment 440 of lower valve housing and then into cylinder 190b. Urging of member 527 rearward causes surface 520 to pass under lip 519, whereas spring 528 biases lip 519 in front of lip 536 thus holding stored energy 525 in preparation for activation.

Cap 283, connected by tether 280 secured to housing by tab 284 thru orifice 284a, can be removed from flange cap 419 using pull-tab 282, before or after preparation for use. To activate the device 100, a user or healthcare provider pushes on button 105 in a direction generally perpendicular to the longitudinal axis A-A as shown by arrow 198, causing lip 519 to deflect upward such that lip 536 of surface 520 is free to travel forward past and under lip 519 urged by released energy of stored energy 525, sending piston 530 forward in cylinder 190b generally parallel to the longitudinal axis A-A and in the direction of one-way dispensing valve 407, causing the amount medicament to exit flange cap 419.

In one aspect, one-way flow control valve 407 is a duck-bill valve with a predetermined cracking pressure of between about 0.2 to about 1.5 pounds per square inch (psi) (about 1379 to about 10,340 newtons/square meter). Other cracking pressures can be chosen. Other one-way flow control valves can be used. The one-way flow control valve 407 provides for an aseptic delivery system. Biasing member 528 repositions user actuating button 105 after activation.

After dispensing an amount of liquid from the device 100, the device returns to the second configuration described above. Thus, device 100 provides a “dual-action” mode of operation where the device is first placed in a first state by the user by pulling back on ring member 107 and charging the device with an amount of medicament from medicament container 99. Dispensing of the amount of medicament requires the pressing of user-actuating button 105 in a separate action, which places the device in a dynamic state whereby the stored energy is released to piston-cylinder assembly expelling the amount of medicament. Prior to first use, the user may purge the system or may squeeze medicament container 99 to urge medicament into reservoir 414 and/or fluid compartment 440 and/or flow channel 190 or fluid flow path 190a.

Device 100 further includes, as an optional feature, a mechanism or “gravity stop” provided to prevent the device from attempting to withdrawal fluid from the medicament container 99 unless or until the device is properly oriented for the fluidic system to access the liquid contained in the medicament container 99. If not properly oriented, device 100 may allow the introduction of an amount of air into the cylinder 190b when member 527 is drawn back for priming of the device, as one or more side opening 470 may not be in contact with liquid in medicament container 99. By “properly oriented” it is at least meant that the at least one side opening 470 of sheath 480 is in the liquid or below the surface 94, defined by longitudinal axis F-F, of the liquid within the container 99, such that the one or more of side opening 470 has access to the liquid or would otherwise draw air into the cylinder.

Thus, as shown in FIGS. 11-12, ball 580 is floatably positioned in cavity 581 of housing 101, the cavity sized to receive the entirety of ball 580. If device 100 is not properly aligned, at least a portion of ball 580 can enter a socket 582 in member 527, where the socket 582 is slightly larger than cavity 581 so as to readily receive ball 580, and of a shallow depth of no more than half the diameter of ball 580, so that when member 527 is moved such that there is alignment of cavity 581 and socket 582, at least a portion of ball 580 can be received in socket 582 with at least a portion of the ball protruding from the socket thus restricting member 527 and piston 530 from advancing and withdrawing fluid from medicament container 99. If the device 100 is properly oriented, ball 580 is not introduced into socket 582 even though cavity 581 may be aligned with socket 582.

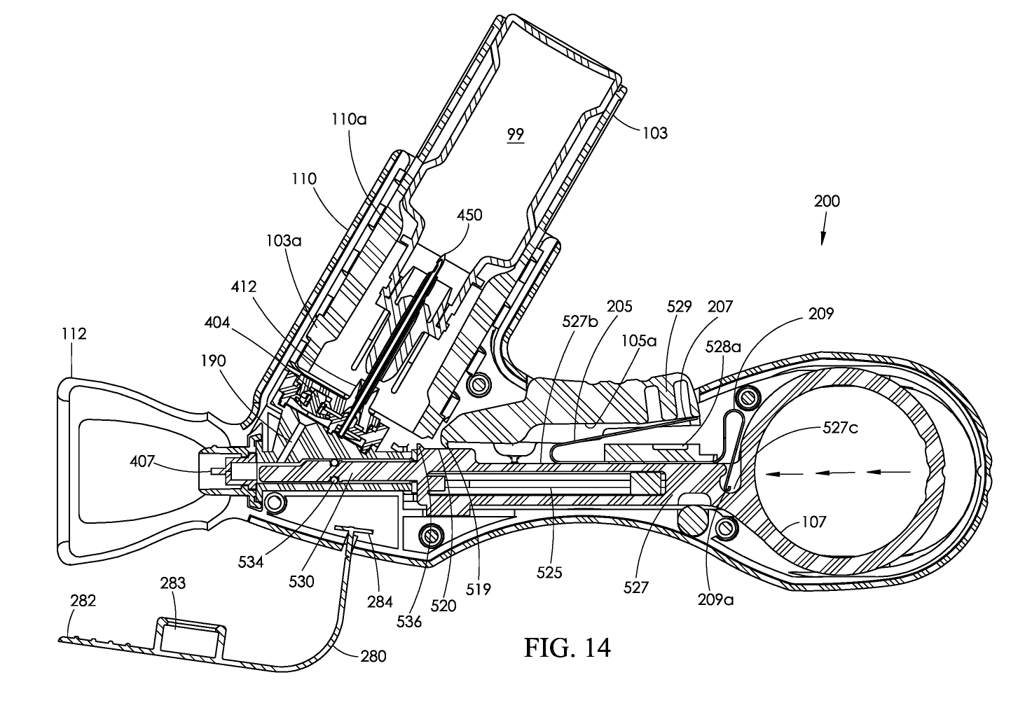

FIG. 14 depicts a sectional view of FIG. 8A along section line 12-12 with an alternate activation embodiment whereby biasing member 528 is replaced with flexible member 205 capable of storing energy, flexible member 205 having a first end 207 in proximity to surface 105a. Opposite end of flexible member 205 is secured under member 528a and cavity 209a in contact with surface 527b. Member 205 can have bends 209 and be contained in housing in cutout 527c All other operational, functional, and structural elements being the same as previously described for device 100.

Other activation systems may be employed with the carriage member/carriage receiving member and fluidic system described above for device 100, for example, as disclosed in PCT application number PCT/US2015/058855, incorporated herein by reference in its entirety.

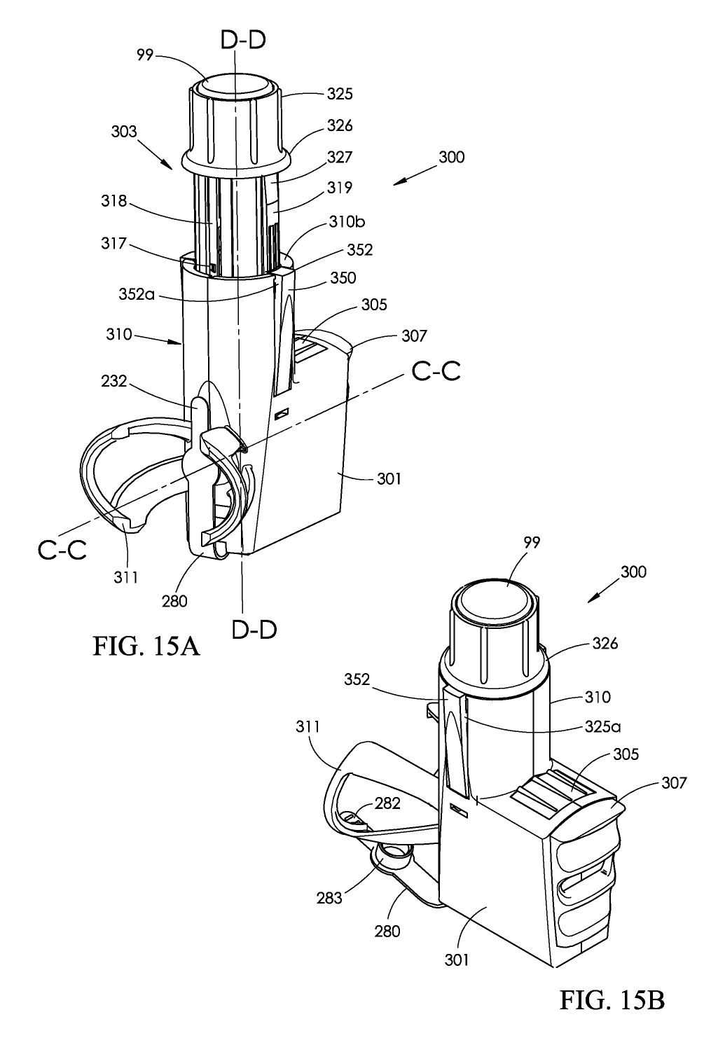

A second embodiment of the device of the present disclosure is provided in FIGS. 15A-43 is now described. In the second embodiment, a modified carriage member and carriage receiving member are provided. While aspects of the piston cylinder arrangement of the first embodiment are maintained, the second embodiment provides for a modified actuating system, where the piston is drawn back using a perpendicularly directed force rather than a parallel directed force. The second embodiment also uses a “dual-action” mode of operation, where the device is first placed in a first state by the user by providing a perpendicular force relative to the longitudinal transitioning of the piston in the cylinder for charging the device with an amount of medicament from medicament container 99.

Dispensing of the amount of medicament requires the pressing of user-actuating button 105 in a separate action, which places the device in a second dynamic state whereby the stored energy is released to piston-cylinder assembly expelling the amount of medicament. In addition, the second embodiment may provide a device suitable for one-handed operation and control and having less total material and a smaller footprint. Such a configuration may prevent or eliminate accidently giving or receiving a “double dose” as each dose has to be loaded/primed by pulling the piston back (directly or indirectly) before it can be delivered with an activation event, as each dose has to be manually chambered with a separate action.

FIG. 15A and FIG. 15B depict perspective views of the second embodiment of the present disclosure depicting device 300 and an initial state and a first state, respectively, having cap 325 of carriage member 303 cooperatively engaged with carriage manipulation system comprising a carriage member 303 and carriage receiving member 310. Carriage receiving member 310 is shown projecting from housing 301 essentially perpendicular to the longitudinal axis C-C. Housing 301 is shown as components 301a and 301b joined together to form housing 301. Carriage member 303 is provided in an initial state partially threaded with carriage receiving member 310 along longitudinal axis D-D. Slider 307 cooperates with button 305 to prime the device 300 with an amount of liquid from medicament container 99 and to activate the device for dispensing the amount of liquid. Slider 307 and button 305 transverse essentially parallel to the longitudinal axis D-D during normal operation. Optional positioner 311, cap 283, tether 280, and distal end 282 are structurally and functionally equivalent to that described above for the first embodiment.

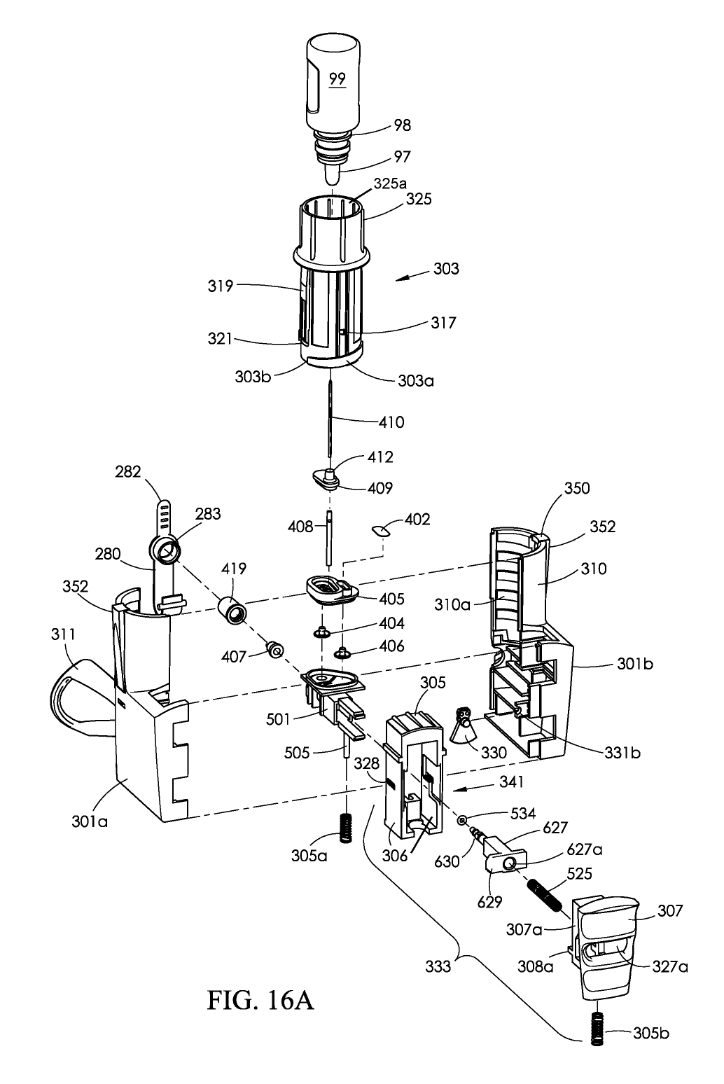

With reference to FIG. 16A, which depicts an exploded view of the device 300 of FIG. 15A, as well as FIG. 16B, which depicts a partial exploded view of the activation mechanism, fluidic system and housing of the present disclosure, medicament container 99 with tapered nozzle 97 is configured to be received by opening 325a in cap 325 of carriage member 303.

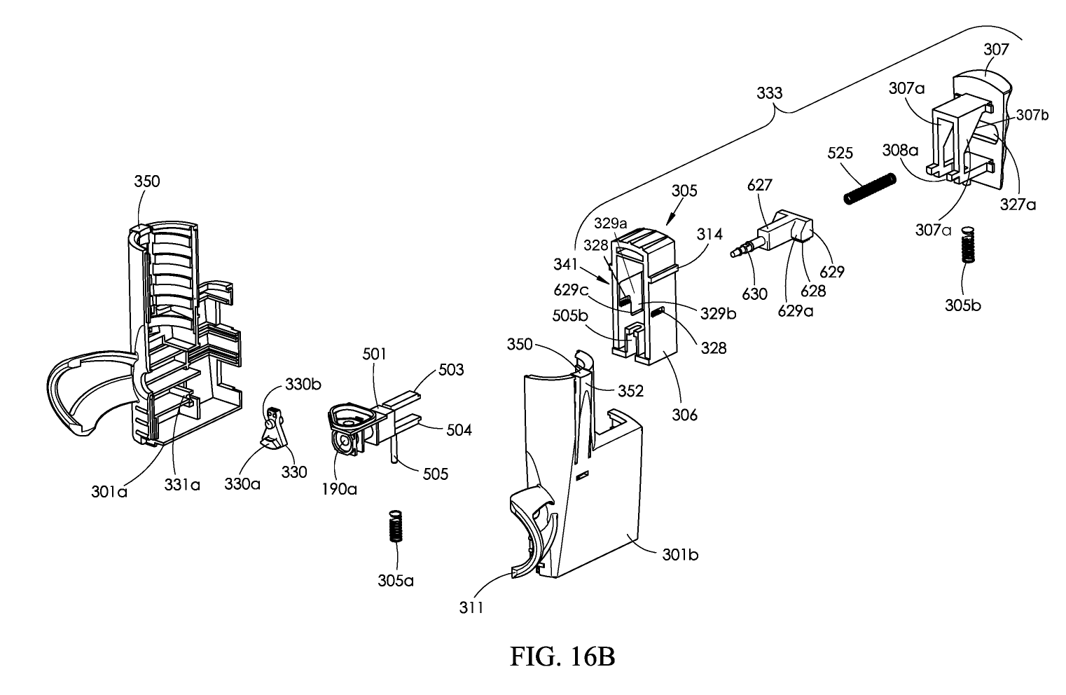

With reference to FIG. 17, an exploded view of a fluid flow system 400a of the present disclosure is shown, where cannula 410, sheath 408, seat 412, flange seal 409, upper valve housing 405, filter 402, filter housing 402a, valve 407, flow control valves 404, 406, ferrule 413, fluid reservoir 414, coupling 406a, and flange seal cap 419, are structurally and functionally equivalent to that as described above as in FIG. 2. Device 300 comprises a lower valve housing 501 with vent compartment 442, fluid compartment 440, partition 441, and flow channel 190 and flow path 190a as described above as in FIG. 2. Lower valve housing 501 has parallel arms 503, 504 extending from housing 501 essentially parallel with longitudinal axis C-C and the piston 630-cylinder 190b path. Arms 503, 504 are received by button 305 between parallel sidewalls 306, and the arms are positioned above and below member 627, respectively, and are received by slider 307 between a pair of extending parallel surfaces 307a.

Referring now to FIG. 16B and FIG. 17, post 505 extends perpendicular to the longitudinal axis C-C and receives spring 305a. Parallel sidewalls 306 of button 305 comprises guide track 341 configured to receive a portion of opposed projecting element 629 of member 627. Guide track 341 can be a cutout and/or opening in parallel sidewalls 306. Guide track 341 can be stepped, with an upper region 329a and a lower region 329b parallel to the longitudinal axis C-C, as shown, the upper region includes engagement teeth 328 for receiving corresponding teeth 628 of lower surface of projecting element 629 and lower region 329b without engagement teeth. In one aspect, guide track 341 comprises a vertical surface 629c perpendicular to longitudinal axis C-C separating the upper region 329a and lower region 329b of the guide track 341.

Slider 307 receives projecting elements 629 such that cam following surfaces 629a engage with corresponding cam surfaces 307b of parallel surfaces 307a when button 305 is traversed (in a direction essentially perpendicular to longitudinal axis D-D and the piston 630-cylinder 190b axis). In one aspect, slider 307 receives projecting element 629 such that cam following surfaces 629a engage with corresponding cam surfaces 307b when slider 307 is traversed in only one direction (e.g. down) so as to urge the piston rearward in the cylinder so as to draw fluid from the medicament container 99, as discussed further below. Slider 307 is designed to accommodate one or more digits of a human hand to facilitate its operation of being biased downwardly (e.g., in a direction perpendicular to the piston-cylinder longitudinal axis corresponding to longitudinal axis C-C and in a direction away from carriage member 303).

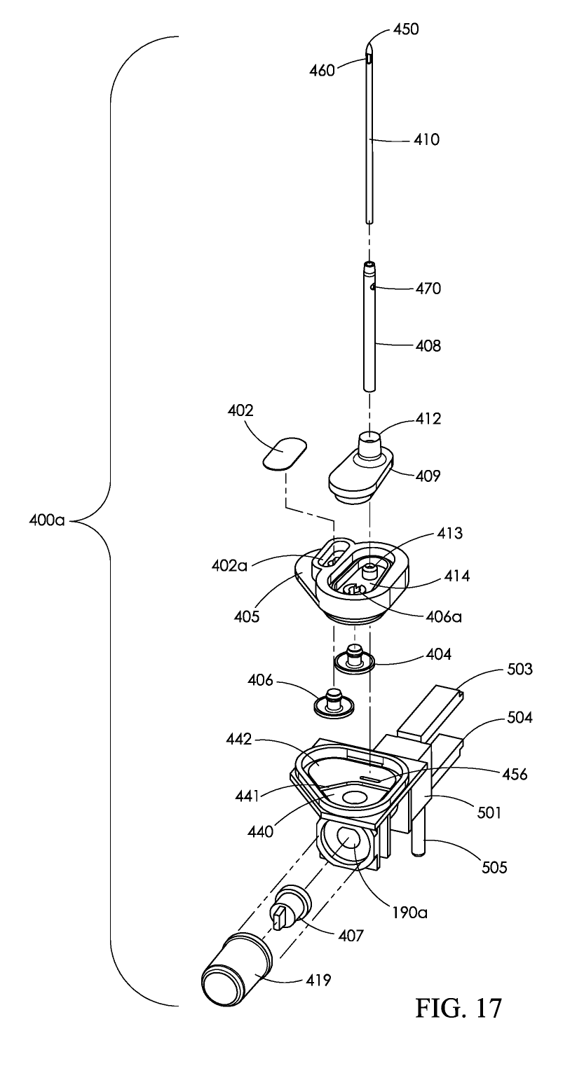

FIG. 18A and FIG. 18B depict perspective views, and FIG. 18C and FIG. 18D depicts a top view, and a bottom view, respectively, of a container manipulation system of the present disclosure comprising a container carriage member 303. Carriage member 303 provides for retention and proper alignment of tapered nozzle 97 of medicament container 99 with distal penetrating end 450 of cannula 410. Opening 325a in cap 325 receives medicament container 99 with tapered nozzle 97 pointing generally towards housing 301. Carriage member 303 comprises male thread segment 303a, and at least one edge surface 303b at either end of the male thread 303a. Male thread 303a is configured for engagement with female threads 310a of carriage receiving member 310. Carriage receiving member 310 provides for, upon assembly, retention of carriage member 303 by way of engaging male thread 303a of carriage member 303 with female threads 310a of carriage receiving member 310 and lip about inner diameter opening of carriage receiving member.

One or more tabs 315 of each of the locking members 318 are positioned between the opposing ends of carriage member 303 and each project inwardly into the interior of carriage member 303, for receiving and retaining annular collar 98 of container 99. During insertion of medicament container 99, locking members 318 and tabs 315 deflect outwardly. Tabs 315 of locking members 318 are shown with angled surfaces on a container receiving side proximal to the opening of cap 325 so as to facilitate deflection of the locking members 318 upon engagement with annular collar 98 of medicament container 99 during insertion into the carriage member 303, and flat surfaces on the opposing side to engage and retain annular collar upon restoration of the deflection of the projecting tabs 315 so as to retain medicament container 99 in the carriage member 303.

Carriage member 303 comprises at least two elongated container locking members 318 that are arranged approximately 180° apart about the outer perimeter of carriage member 303 projecting parallel to longitudinal axis E-E. Locking members 318 each terminate at tabs 315 distal from male thread 303a in proximity to cap 325. Locking members 318 are provided in an initial configuration generally unbiased, but are configured to deflect slightly outward from the outer diameter of carriage member 303 during insertion of medicament container 99, and to essentially return to the initial configuration. One or more tabs 315 of each of the locking members 318 are positioned between the opposing ends of carriage member 303 and project outwardly for receiving and retaining annular collar 98 of container 99. Inwardly projecting tabs 315 of locking members 318 are shown with angled surfaces on one side proximal to the opening of cap 325 so as to facilitate deflection of the locking members 318 upon engagement with annular collar 98 of medicament container 99 during insertion into the carriage member 303, and flat surfaces on the opposing side to engage and retain annular collar upon restoration of the deflection of the projecting tabs 315 so as to retain medicament container 99 in the carriage member 303. One or more anti-reverse tabs 317 are arranged on outer perimeter of carriage member 303, shown projecting outwardly from surface of locking members 318 and outer perimeter of carriage member 303 (FIG. 18A, 18B).

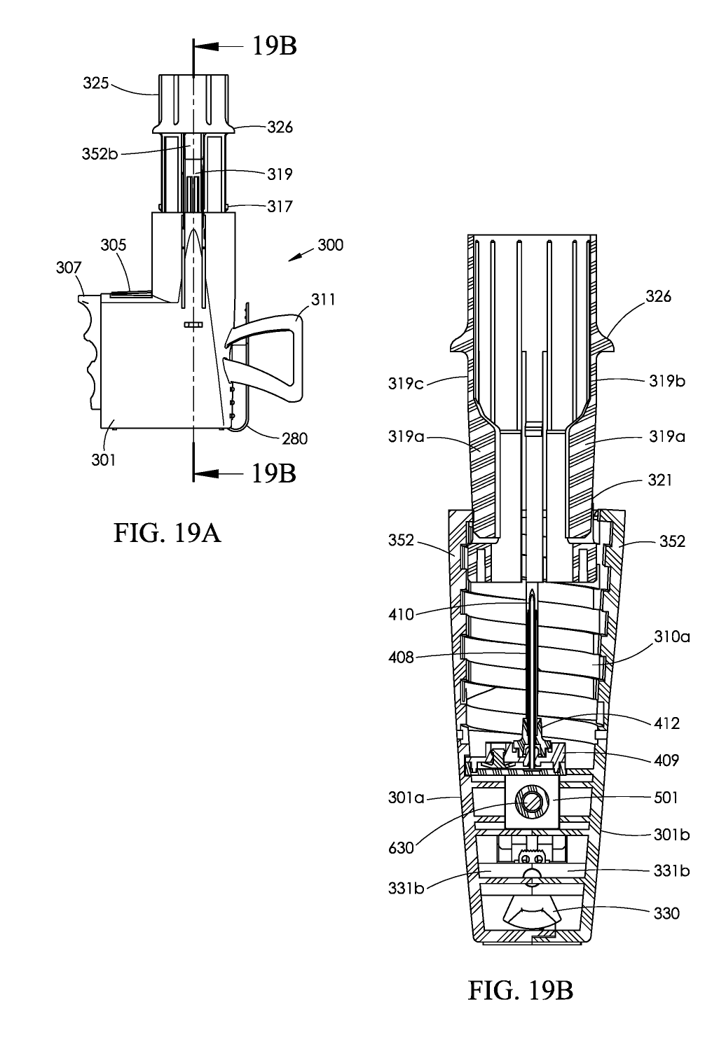

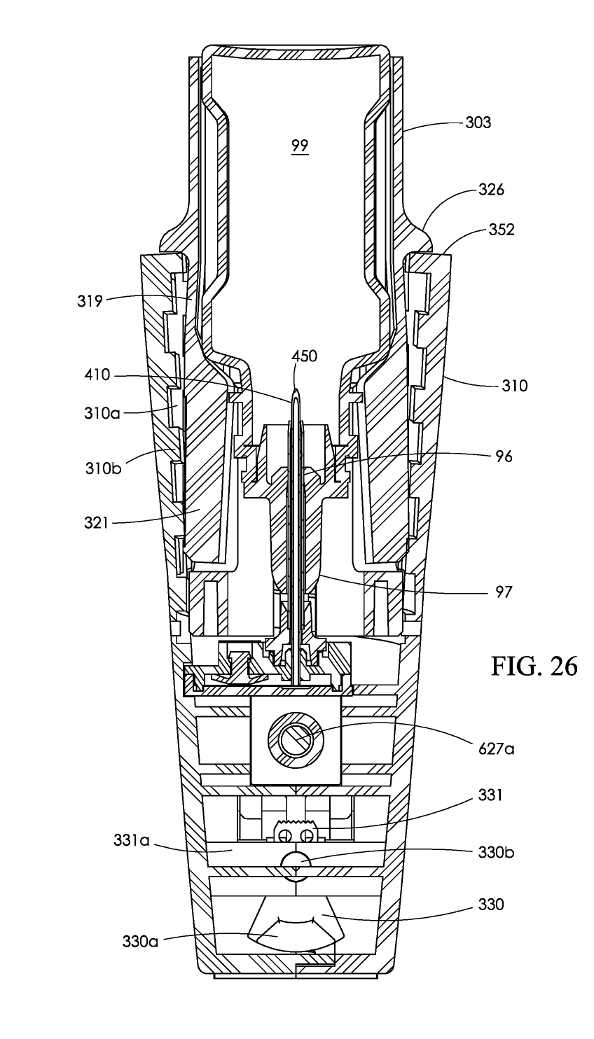

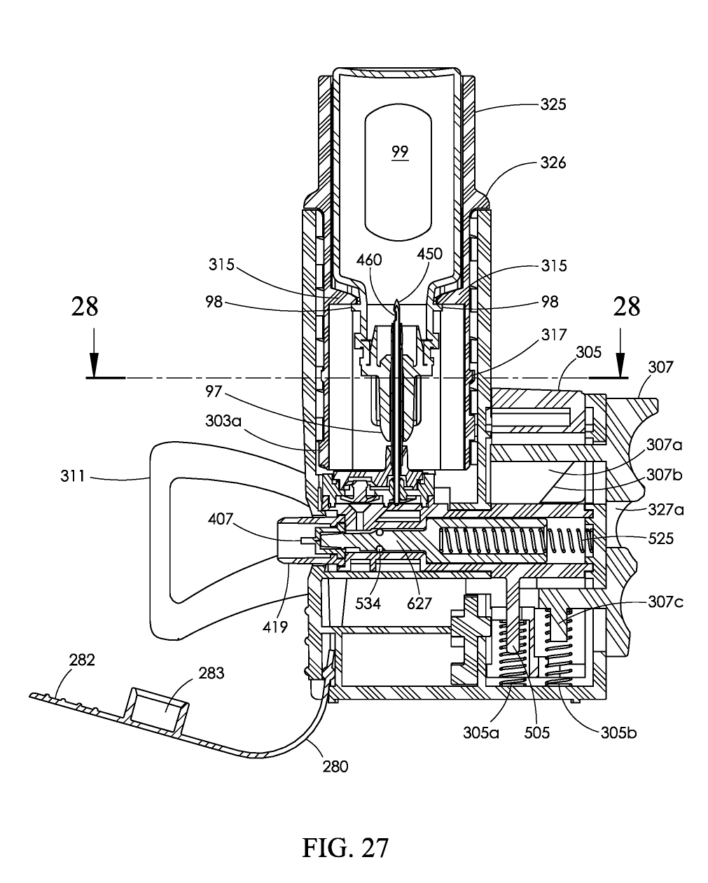

With reference to FIG. 18A, FIG. 18B, FIG. 18C and FIG. 18D, as well as FIG. 28, which is a sectional view of the device 300 of FIG. 27, and top plan view FIG. 20A, anti-reverse tabs 317, configured for preventing reverse rotation of the carriage member 303 from carriage receiving member 310, are shown. Tabs 317 are positioned on the outer perimeter of carriage member 303 between male thread 303a and lip 326 of cap 325. With reference to FIG. 28, Tabs 317 are exemplary shown as projections having a tapered surface 317a and a flat surface 317b, the tapered surface configured to slidably engage female threads 310a of carriage receiving member 310, and the flat surface configured to prevent reverse rotation of the carriage member 303 by engaging a surface between female threads 310a. Tabs 317 are shown on locking members 318, but alternatively can be positioned about the outer perimeter of carriage member 303.

Carriage member 303 further comprises at least two elongated deflecting members 319 arranged approximately 180° apart within a cavity formed through carriage member 303, and the deflecting members are arranged parallel to longitudinal axis E-E, and are shown approximately 90° apart from elongated locking members 318. Other spatial arrangements of the deflecting members 319 and locking members 318 about the carriage member 303 may be employed. Deflecting members 319 each terminate at ends 321 distal from cap 325 in proximity to male thread 303a. Deflecting members 319 are shown having a tapered thickness along their longitudinal length corresponding with longitudinal axis E-E, with a thicker section 319a distal from opening in 325 proximal to end 321. Upon assembly with carriage receiving member 310, at least a portion of the distal end of deflecting members 319 are deflected inward exposing an edge 319b in wall of carriage member 303. Edge 319b is configured to engage edge 352a of projections 352 in the absence of medicament container 99 and prevent rotation (clockwise or counterclockwise). In one aspect, thicker section 319a is stepped from thin section 319c to engage neck 95 of medicament container 99 to assist in the deflecting of end 321 outwardly from the outer perimeter of carriage member 303 and engage and deflect projections 352 of flexible member 350 of carriage receiving member 310, so as to deflect projections 352 and members 350 outwardly from carriage receiving member. Members 350 are proximal to housing 301 and project along axis D-D and distally terminate at opening 310b of carriage receiving member 310. By deflecting projection 352 and members 350 upon insertion of container 99, edges 352a of projections 352 do not engage edges 319b of carriage member 303 during rotation of carriage member 303 allowing male thread 303a of carriage member 303 to fully engage and thread with female threads 310a of carriage receiving member 310.

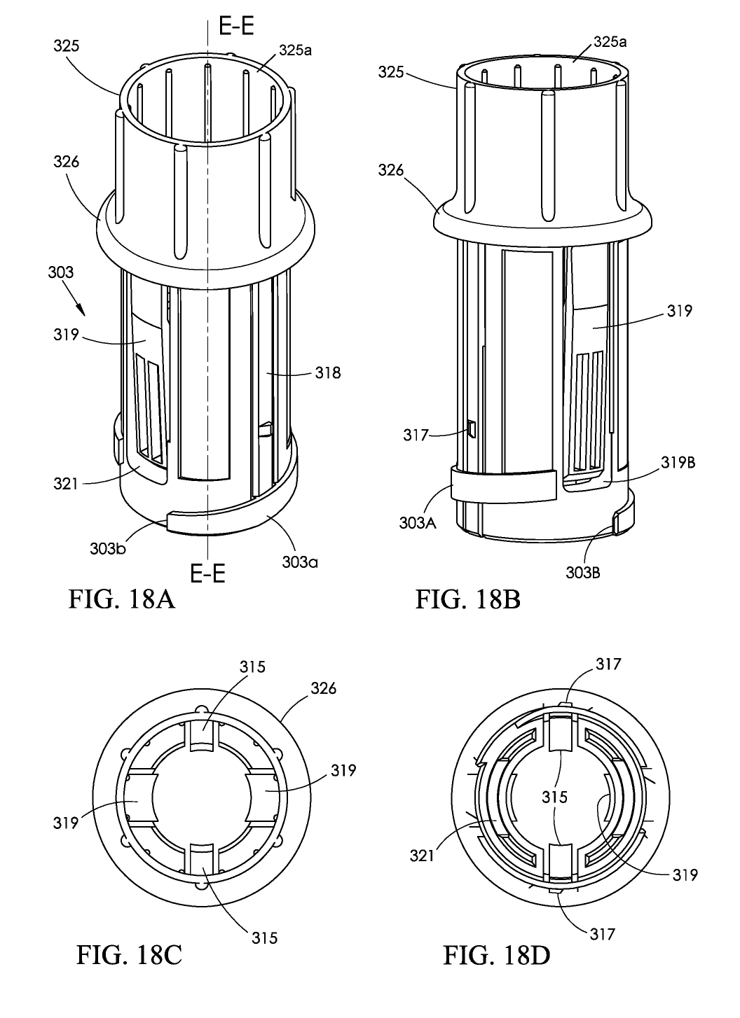

With reference to FIG. 19A, depicting a side view of the device of FIG. 15A, and FIG. 19B, depicting a sectional view along section line 19A-19A of the device of FIG. 19A, the arrangement of carriage member 303 and carriage receiving member 310 are shown in an initial configuration, partially threaded together but otherwise not capable of further threading without an inserted medicament container 99, whereas inwardly projecting members 352 of the elongated members 350 occupy a portion of carriage member 303 between male thread segments 303a preventing the segments from fully threading with corresponding female threads 310a of carriage receiving member.

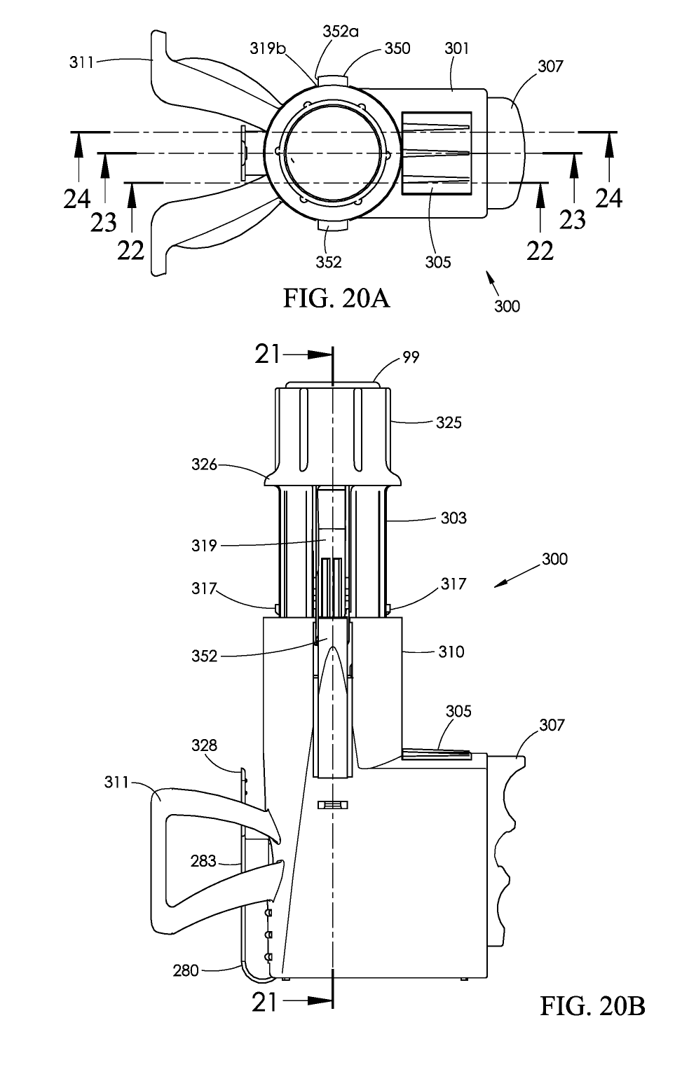

With reference now to FIG. 20B, which is a side view of the device of FIG. 15A, and FIG. 21, which is a sectional view of FIG. 20B along section lines 21-21, the structure and functional relationships between the carriage member 303 and carriage receiving member 310 after insertion of medicament container 99 in carriage member 303 are shown with the container 99 fully received by carriage member 303. Neck 95 of container 99 deflects end 321 of deflecting member 319 which in turn deflects inwardly projecting intrusions 352 of elongated members 350 outwardly from outer perimeter of carriage receiving member 310.

In this first configuration state with the container 99 introduced to carriage member 303, male thread 303a are configured to be fully received by corresponding female threads 310a so as to allow a continuous one-way threading of carriage member 303 with carriage receiving member 310 and to align the opening in tapered nozzle 97 with penetrating and 450 of cannula 410.

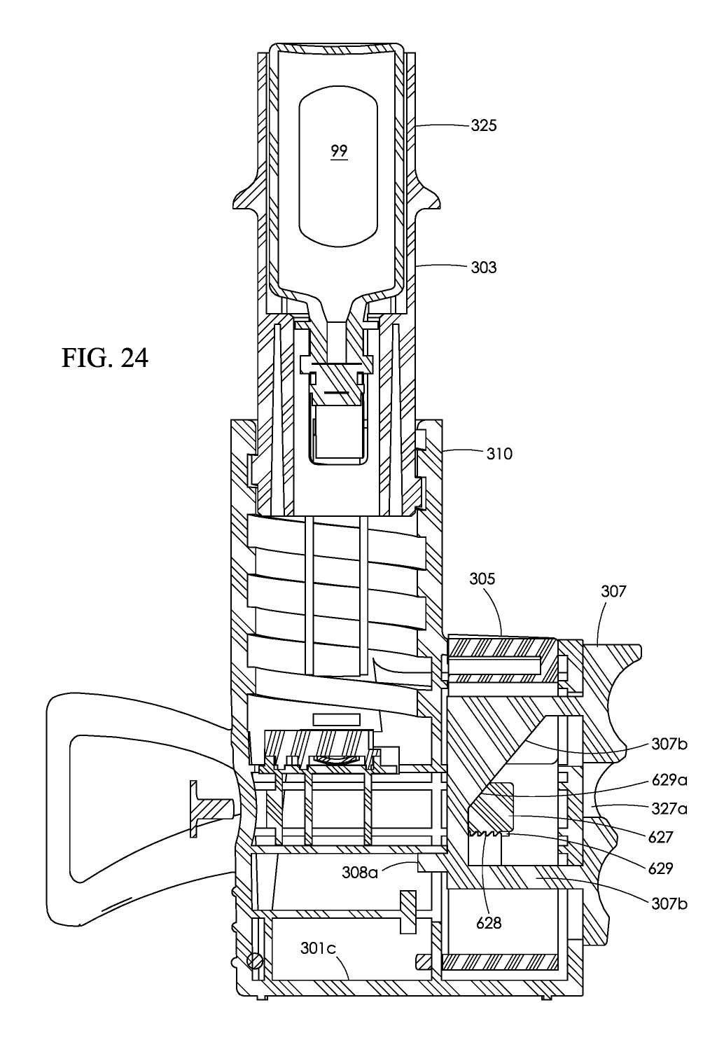

With reference now to FIGS. 25A, 25B, 26 and 27, a second configuration of device 300 is shown, whereby carriage member 303 has been fully threaded with carriage receiving member 310 such that tapered nozzle 97 and opening 96 of medicament container 99 has been properly aligned and penetrated by penetrating end 450 of cannula 410 such that liquid in medicament container 99 is fluidically coupled to fluidic system 400a. FIGS. 20A, 20B, 22, 23 and 24 are similar in presentation to that of FIGS. 25A-27, but with the medicament container 99, and shows that activation system 333 is unchanged by the insertion of the container 99 and the threading of carriage member 303 with carriage receiving member 310. In this configuration, device 300 is in preparation for priming by a user or healthcare professional.

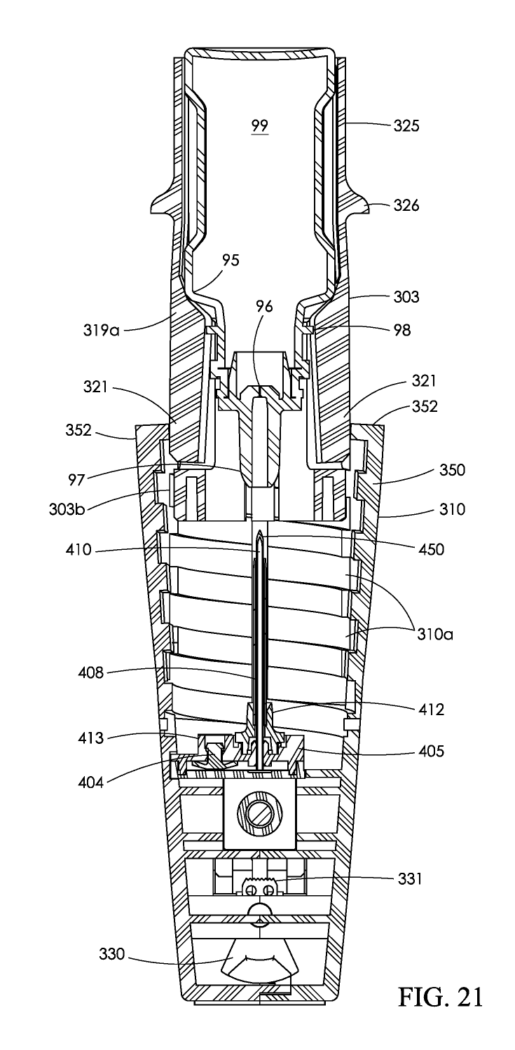

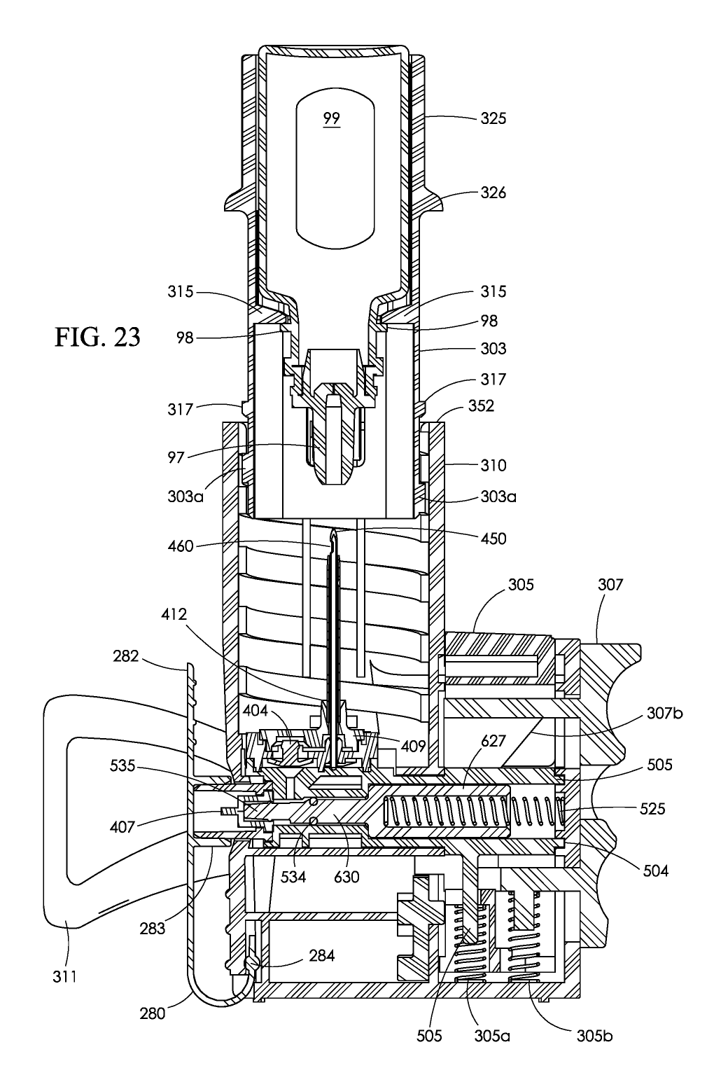

The activation system 333 is now described. With reference to FIGS. 22 and 24, sidewall 306 of button 305 having upper region 329a and engagement teeth 328 cooperatively engaged with corresponding teeth 628 of lower surface of projecting element 629 above lower region 329b of guide track 341, as shown, having upper region with engagement teeth 628 for receiving corresponding teeth 628. A portion of cam following surfaces 629a of element 629 is shown engaged with cam surface 307b.

Other activation systems may be employed with the carriage member/carriage receiving member and fluidic system described above for device 300, for example, as disclosed in PCT application number PCT/US2015/058855, incorporated herein by reference in its entirety.

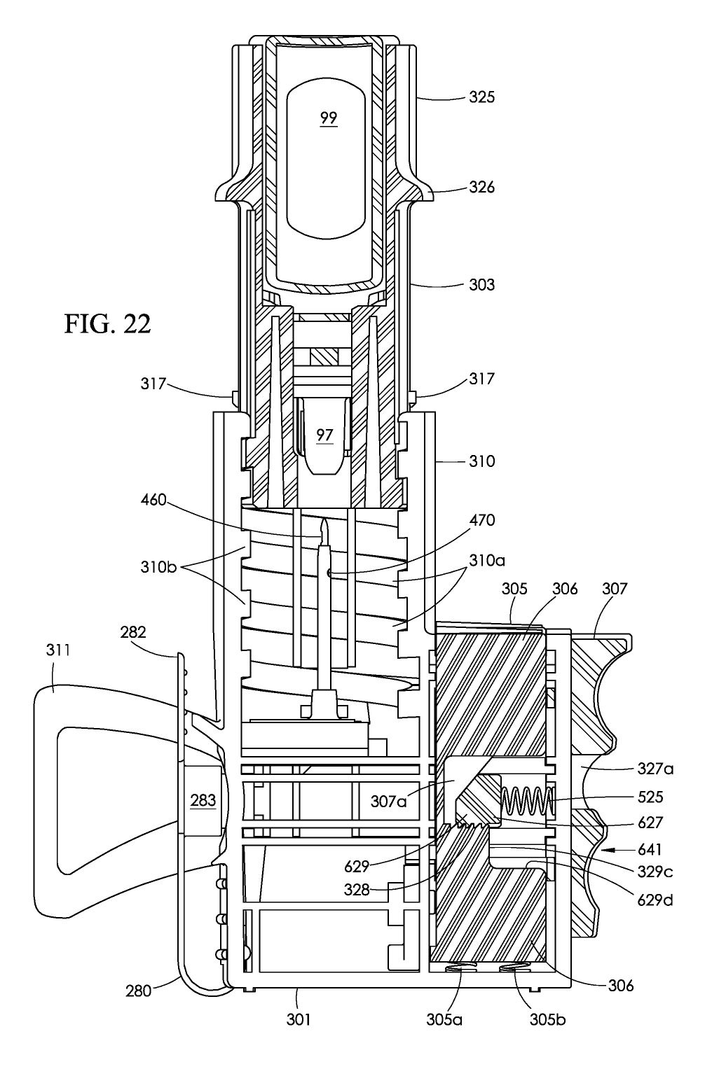

FIG. 23 shows the internal structure of piston 630 positioned between extending arms 505 and 504 of fluidic system so as to guide plunger rod in cylinder in a lateral path parallel with longitudinal axis C-C. Piston 630 is biased by spring 525 secured to housing and contained in the plunger rod and aligned with the longitudinal axis C-C. Post 505 positions spring 305a for biasing distal end of button 305 in reversible paths of motion perpendicular to longitudinal axis C-C and parallel with longitudinal axis D-D. Distal end 535 of piston 630 is shown in a sealing relationship with one-way dispensing valve 407. Distal end 535 configured for sealing with dispensing valve 407 provides aseptic integrity and/or minimizes or reduces contamination of the fluid flow path 190a by minimizing dead volume and/or preventing a backflow of air or vapor into the flow channel.

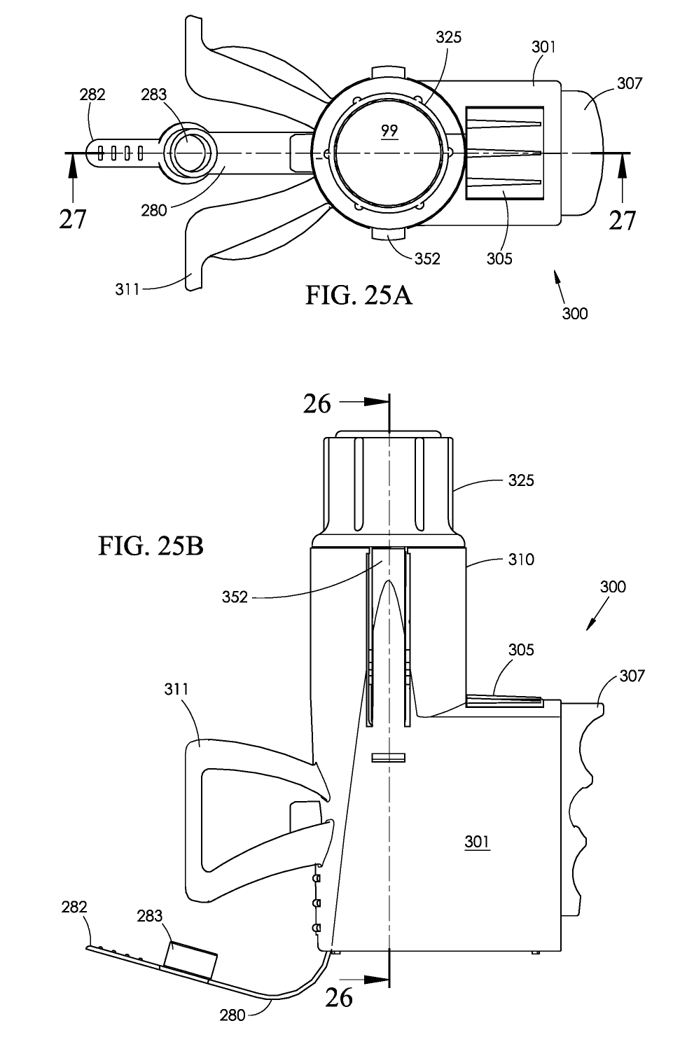

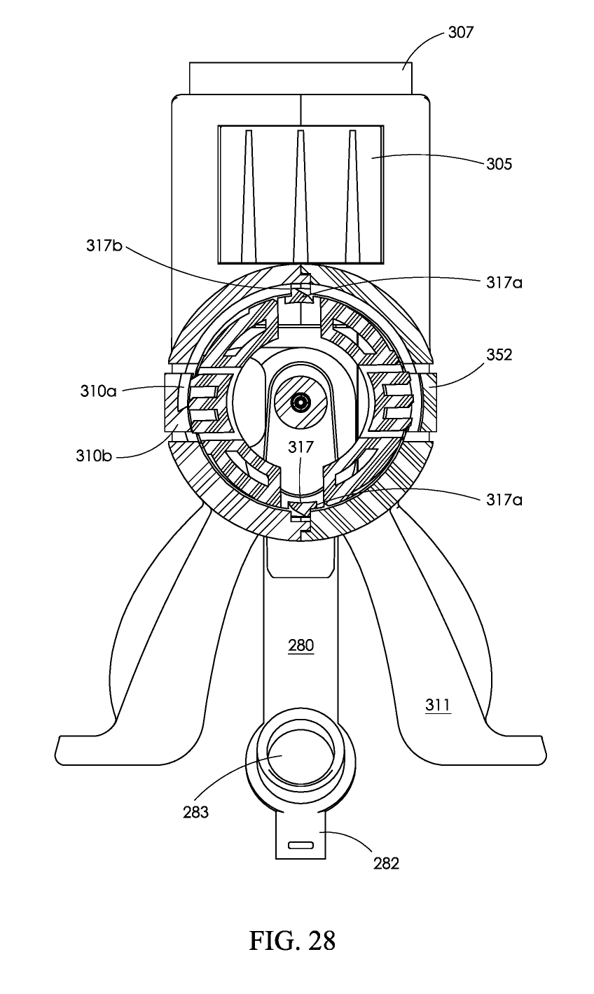

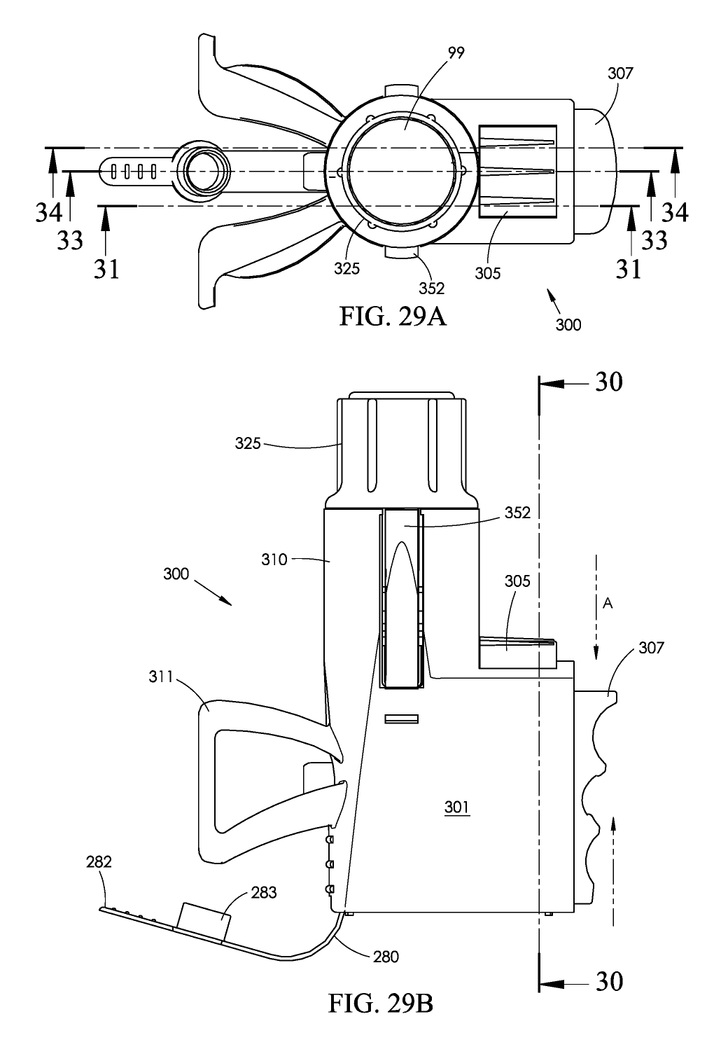

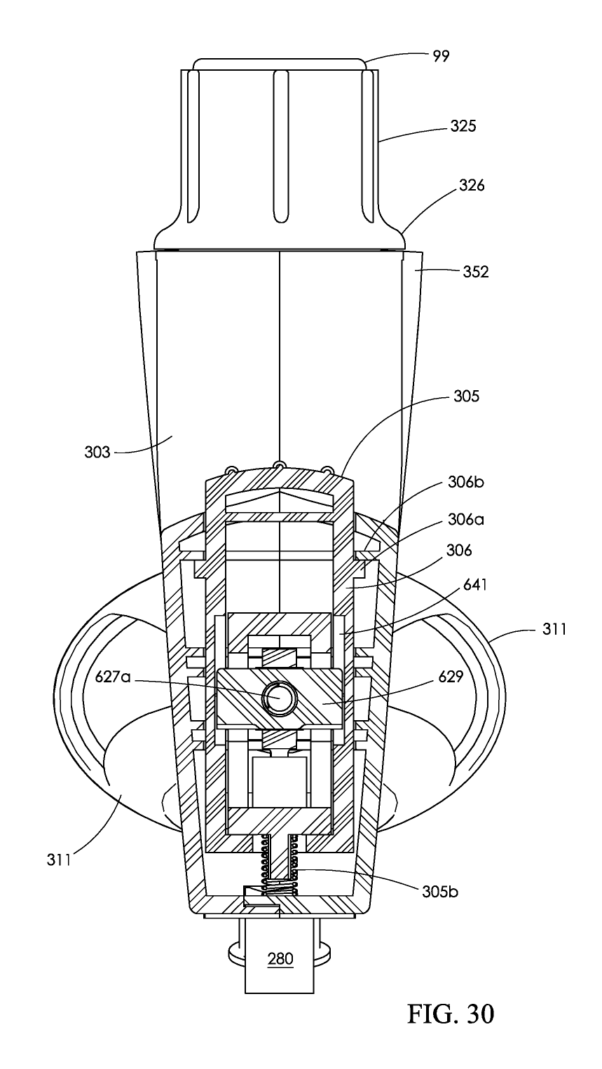

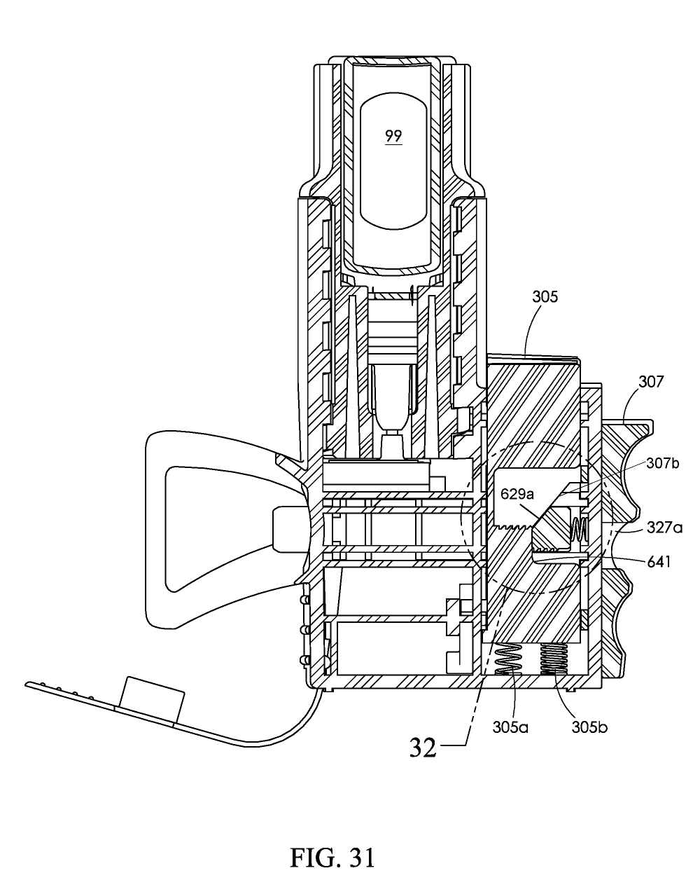

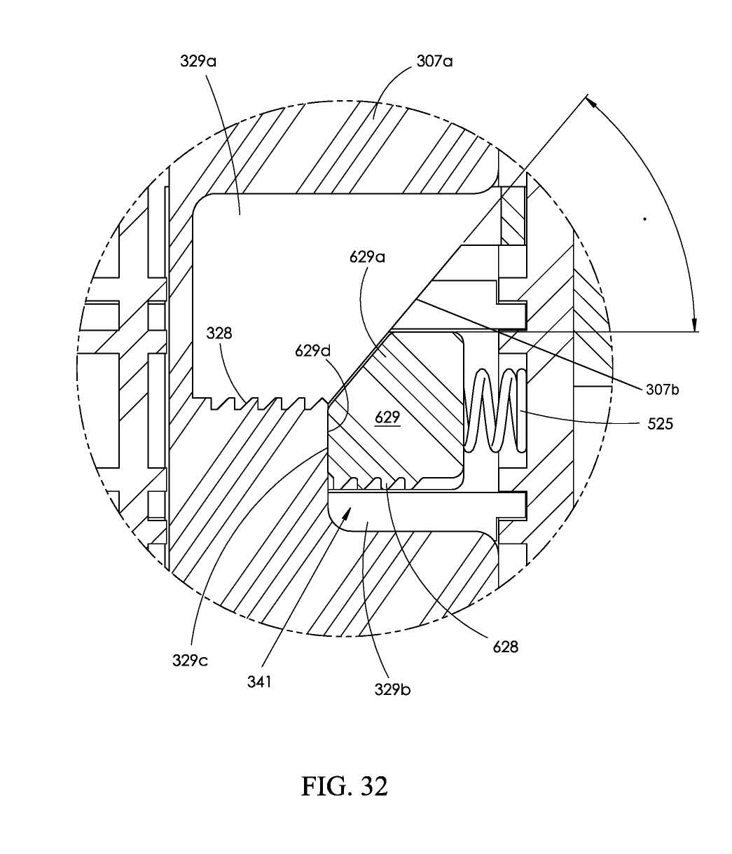

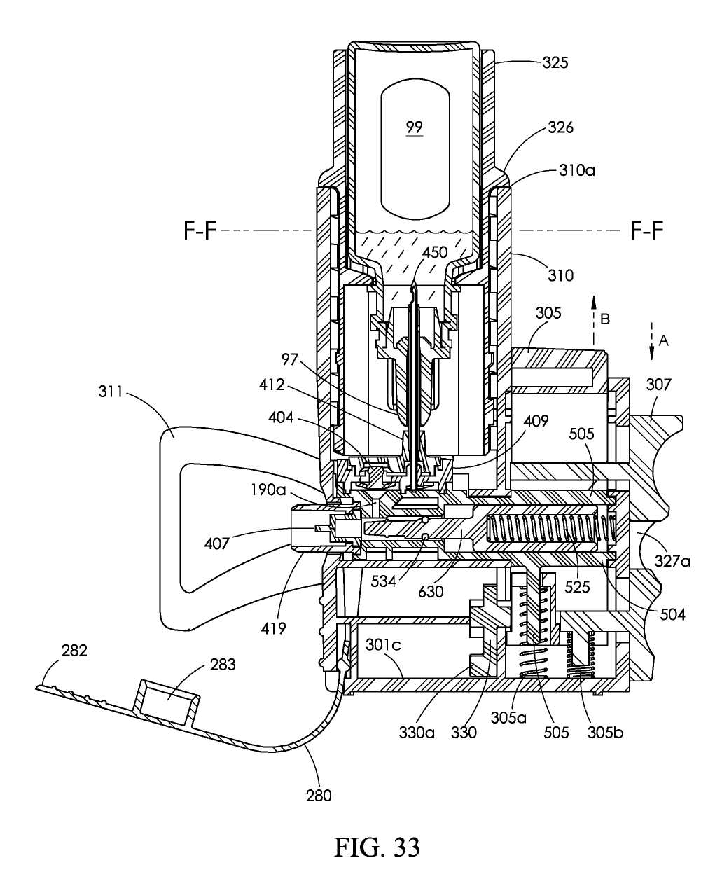

With reference now to FIG. 25A, which is a top plan view of FIG. 15B, FIG. 25B, which is a side view of FIG. 15B, FIG. 26, which is a sectional view of the device of FIG. 25B along section line 26-26, and FIG. 27, which is a sectional view of the device of FIG. 25A along section line 27-27, and with reference to FIG. 29A, which is a top plan view of the device of FIG. 15B, FIG. 29B, which is a side view of the device of FIG. 15B, FIG. 30, which is a sectional view of FIG. 29B along section line 30-30, FIG. 31, which is a sectional view of FIG. 29A along section line 31-31, FIG. 32, which is a partial sectional view of FIG. 29A along section line 32-32, FIG. 33, which is a sectional view of FIG. 29A along section line 33-33, and FIG. 34, which is a sectional view of FIG. 29A along section line 34-34 in a final configuration where the device 300 is in preparation for dispensing (in a pre-activated state) an amount of liquid removed from the medicament container 99 and presented to flow channel 190 and cylinder 190b.

Referring to FIGS. 29B, 31, 32 and 33, to arrive at the final configuration, a user or healthcare provider would apply a force to slider 307 that is perpendicular to the longitudinal axis C-C of the device and in a direction generally away from the carriage member 303 and carriage receiving member 310 in the direction of arrow A. In doing so, cam surface 307b engages cam following surface 629a and withdraws piston 630 against bias from spring 525 from cylinder 190b along a path parallel to longitudinal axis C-C and in the direction of slider 307. Vacuum created in post 505 causes fluid from medicament container 99 to enter side opening 470 of sheath 408 and into fluid reservoir 414, through flow control valve 406 into fluid compartment 443 so as to provide an amount of fluid to enter flow channel 190 and fluid flow path 190a in preparation for dispensing.

Cam surface 307b engages cam following surface 629a until element 629 is urged past vertical surface 329c of guide track 341. Upon clearing vertical surface 329c, button 305 is biased upwardly, arrow B, in a perpendicular path to that of longitudinal axis C-C (a path generally parallel with longitudinal axis D-D and in the general direction of carriage member 303) by spring 305a. Lateral protrusions 314 (FIG. 16B) on opposite sides of button 305 are configured to stop the travel of button 305 against housing elements 301c. Vertical surfaces 329c of button 305 are positioned against vertical surfaces 629d of element 629, which is biased by spring 525 in the 3^{rd} configuration e.g., a primed and loaded configuration ready to dispense an amount of liquid upon activation (depression) of button 305. With reference to FIG. 32, the angle of cam following surface 629a may be optimized so as to facilitate the force needed on slider 307. In one aspect, the angle “alpha” (α) can be between 20 and 60°, or between 30 and 50°, or between 35 to 45°. Likewise, engagement teeth 328 and 629 6 can be configured to maximize engagement during the cam action and to minimize engagement during activation, as discussed below.

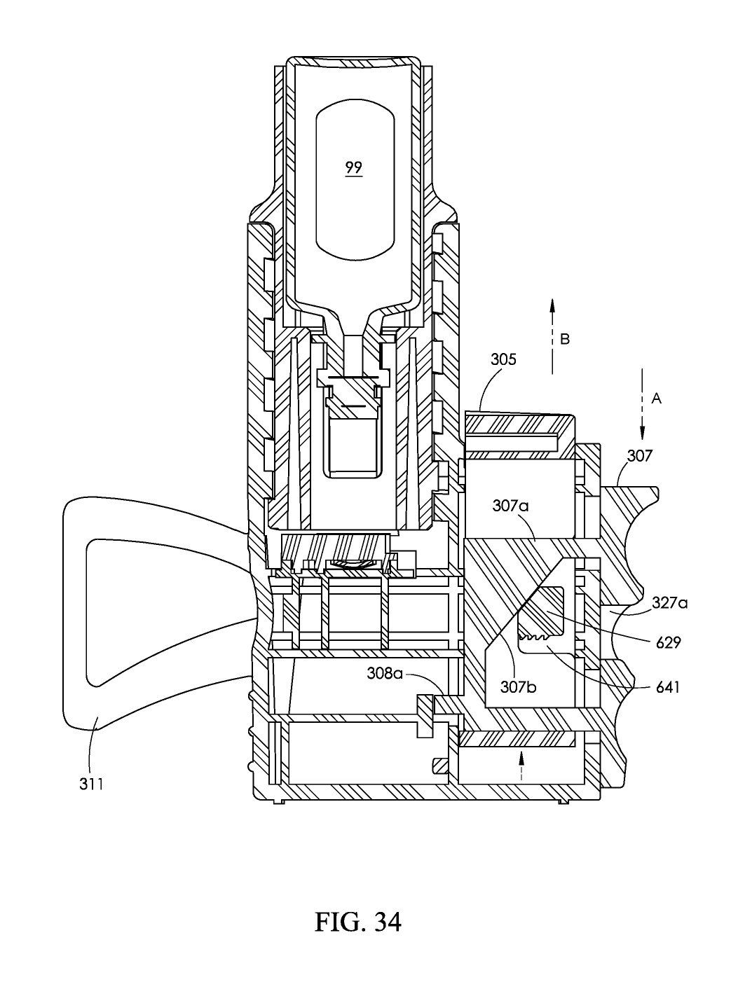

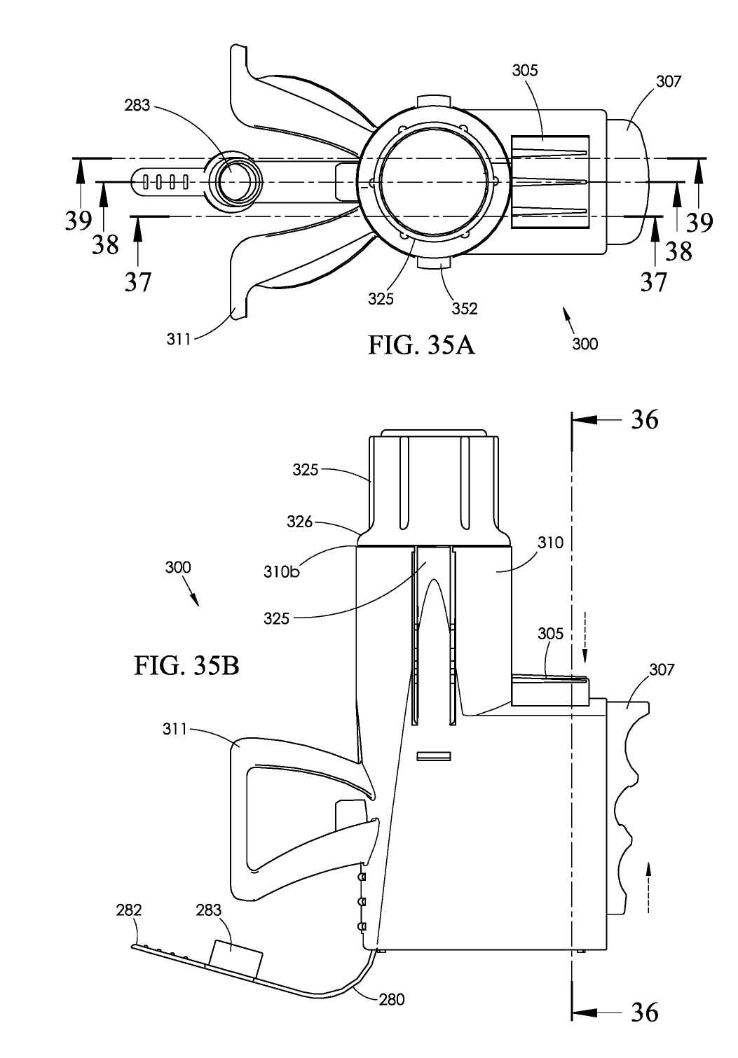

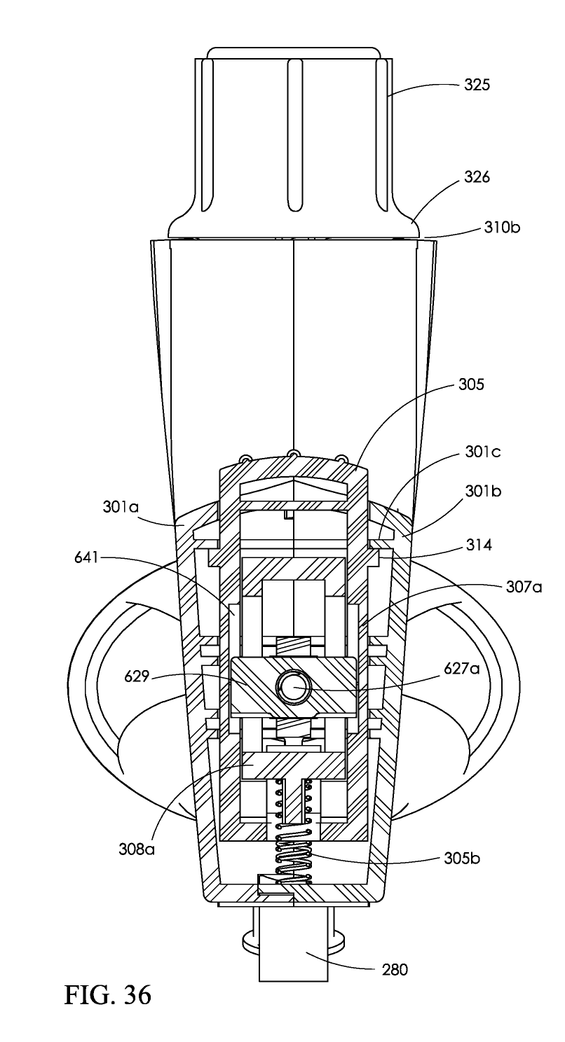

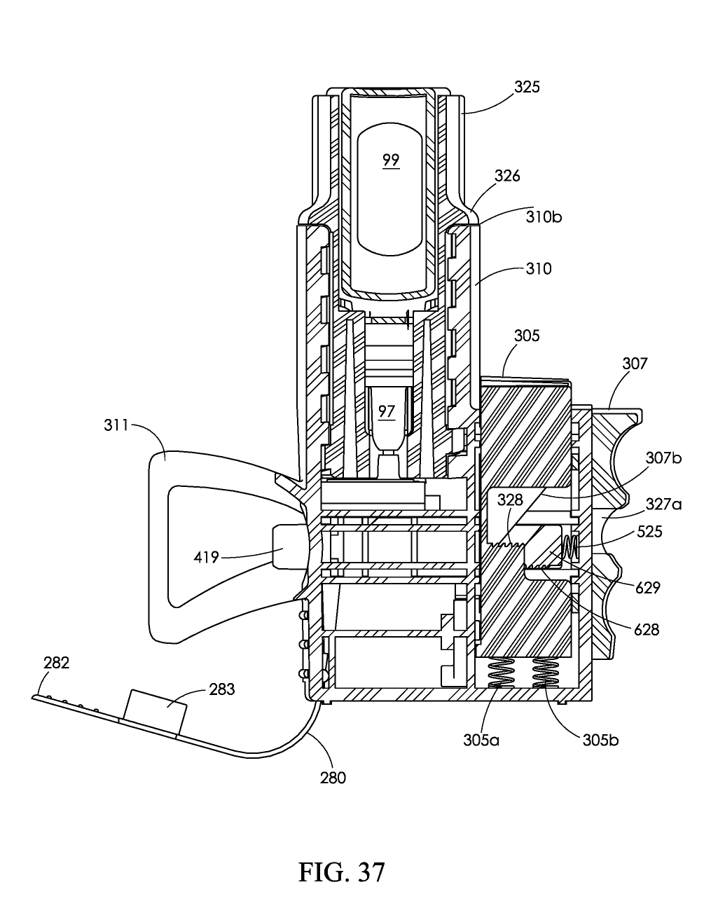

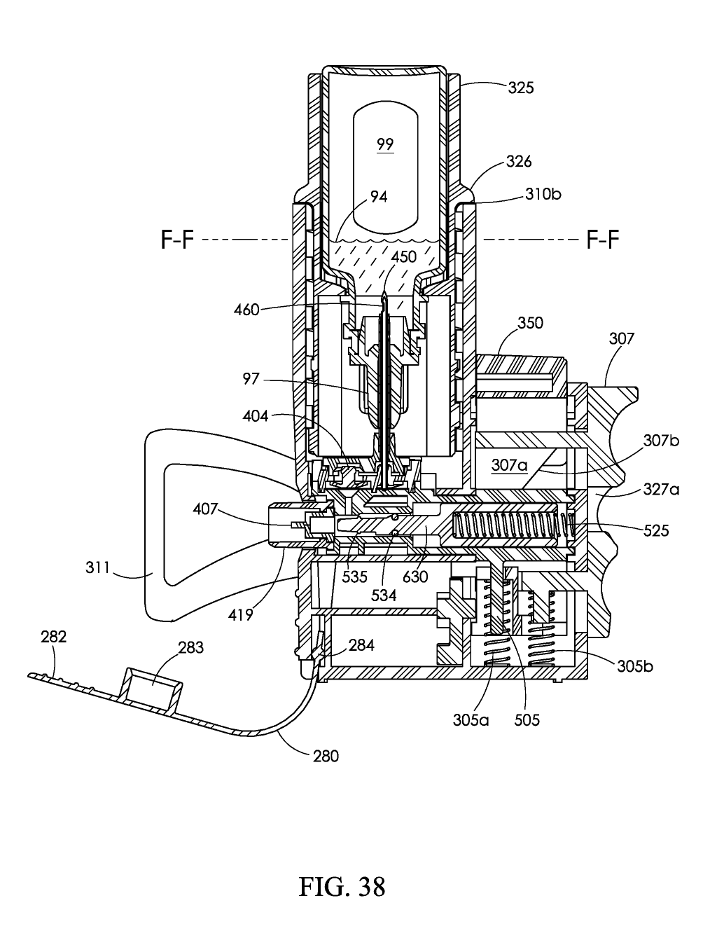

Referring now to FIG. 35A, which is a top plan view of the device of FIG. 15B, FIG. 35B, which is a side view of the device of FIG. 15B, FIG. 36, which is a sectional view of FIG. 35B along section line 36-36, FIG. 37, which is a sectional view of FIG. 35A along section line 37-37, FIG. 38, which is a sectional view of FIG. 35A along section line 38-38, and FIG. 39, which is a sectional view of FIG. 35A along section line 39-39, device 300 has partially withdrawn piston 630 from cylinder containing an amount of liquid for dispensing, piston 630 biased from spring 525 along a path parallel to longitudinal axis C-C and releasable in the direction of one-way dispensing valve 407. Button 305 now protrudes partially from housing 301 and slider 307 has returned to its initial configuration due to bias from spring 305b. In one aspect, the side surfaces of button 350 that are protruding from housing 301 may be brightly colored (e.g., fluorescent red, green, or orange) so as to indicate to the user or healthcare provider that device 300 is primed and activated for dispensing amounts of fluid from medicament container 99.

As an advantage, device 300 is configured such that the user cannot actuate the device or be given an indication that the device has been activated until a sequence of steps are performed. This is to avoid a “false administration event” Small amounts of liquids, especially clear liquids may not be readily visible to the user or healthcare provider to otherwise confirm administration. For example, button 305 is presented in a raised position only when device 300 is primed and an amount of liquid is present in the cylinder 190b for dispensing. Otherwise, button 305 is essentially flush with the housing 301, e.g., a dead button, and does not respond to pushing by the user, in contrast to the primed configuration where button 305 is raised from housing 301. In one aspect, the shape of the surface of button 305 is concave and mostly recessed in housing 301 when the device is not primed.

Upon depression of button 305 in a downward direction, arrow C, (e.g., a path generally parallel with longitudinal axis D-D and in the general direction away from carriage member 303) vertical face 329c is urged passed the engagement teeth 628 of elements 629 releasing the bias from spring 525 and urging the piston 630 in a direction away from slider 307 and a path in the cylinder 190b generally parallel with longitudinal axis C-C so as to dispense the amount of liquid. Engagement teeth 328 and 629 are configured at an angle such that accidental engagement is avoided during activation when the piston 630 is driven towards the dispensing valve 407. After dispensing an amount of liquid from the device 300, the device returns to the second configuration described above.

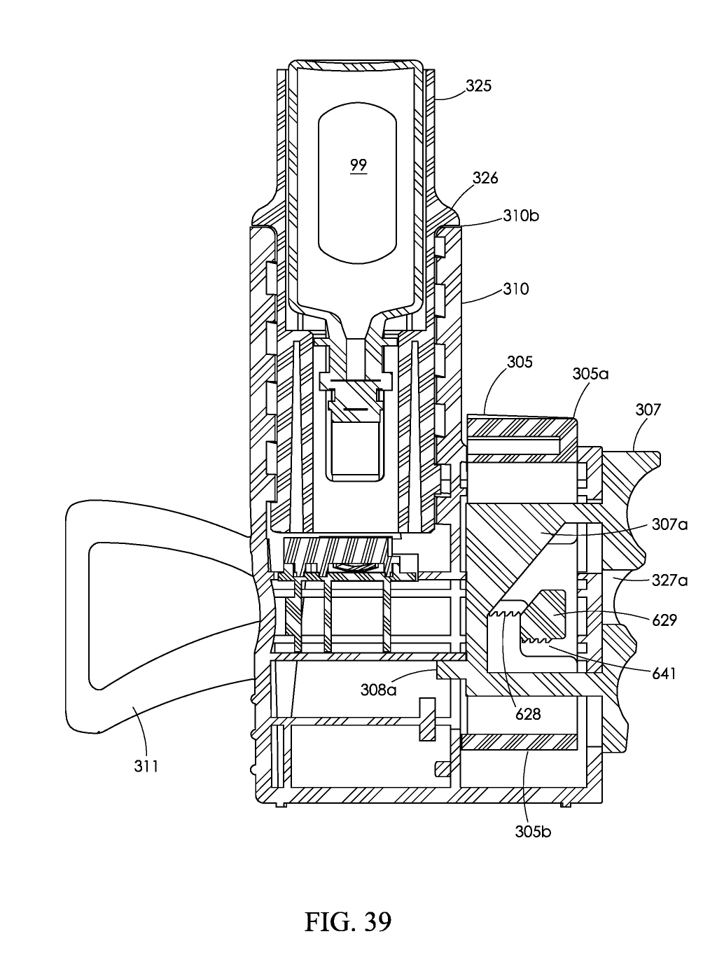

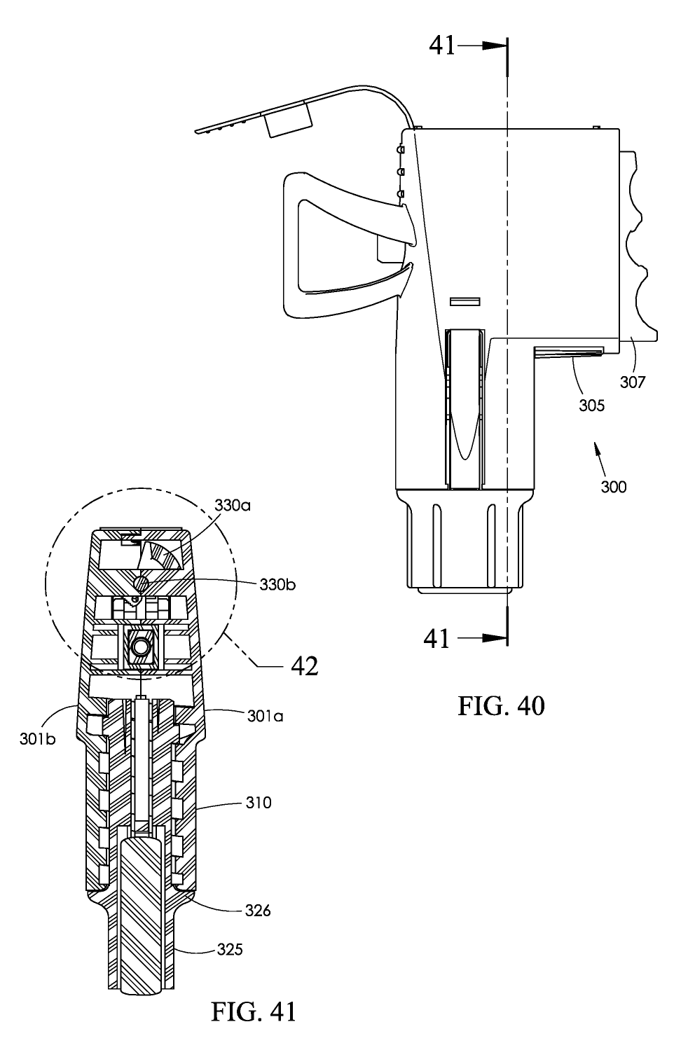

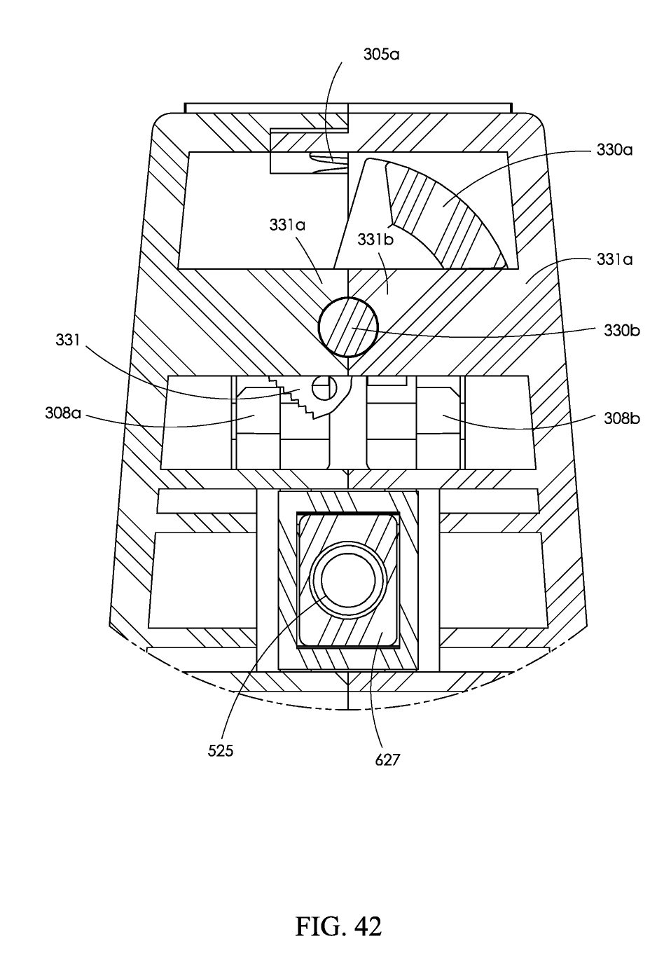

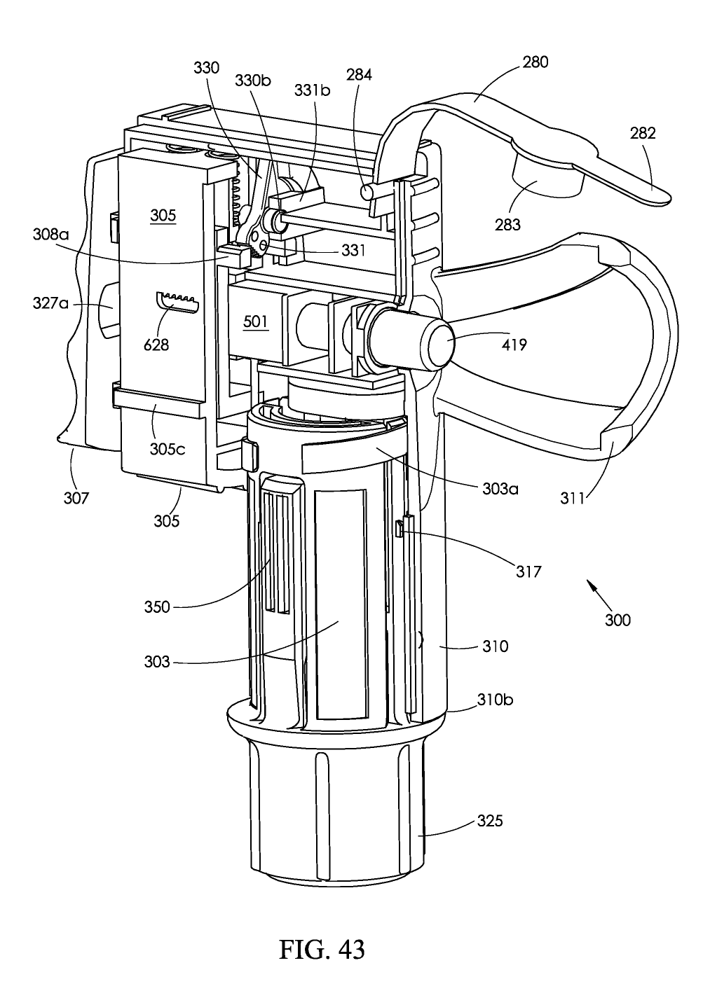

With reference to FIGS. 16A, 16B, 19B, 21, 26, 33, FIG. 40, which is a side view of the device of FIG. 15B in an inverted state, FIG. 41, which is a sectional view of the device of FIG. 40 along section line 41-41, FIG. 42, which is a partial sectional view of section 42 of FIG. 41, and FIG. 43, which is a partial cutaway view of FIG. 40, a gravity break/gravity stop member 330 of device 300 is depicted which functions to prevent the piston 630 from being withdrawn from the cylinder 190b and drawing and air into cylinder 190b when the device 300 is oriented such that the at least one side-port 470 of sheath 480 is above the liquid or the liquid surface 94, defined by longitudinal axis F-F, in the medicament container 99, however, this stop feature member 330 of the present embodiment is structurally adapted for the smaller footprint and modifications of device 300 and that of the activation system 400a. Thus, member 330 which is shown having generally a pendulum shape, has a weighted end 330a and a pivoting member 330b adapted to be configured with housing elements 331a so as to swing freely about pivoting member. A portion of member 330 is configured to have a first configuration where the device 300 is properly oriented as defined above such that protruding members 308a of slider 307 when traveling in a path perpendicular to longitudinal axis C-C are not blocked by member 330. If device 300 is not properly oriented such that member 330 is in a second configuration relative to protruding members 308 of slider 307 such that slider is prevented from fully traveling in a path perpendicular to the longitudinal axis C-C. Various alternative shapes and configurations of member 330 are possible so as to provide a similar function as described.

Thus, the fixed fluidic path dimensions of the above devices 100, 300 are adapted to introduce a medicament liquid of approximately uniform amounts, for example in one or more droplets. The droplets can be of a size in the range 20 to 200 micron in diameter, or can be smaller or larger. The droplet size can be adjusted based on the viscosity of the medicament and the sizing of the exit valve and fluidic system dimensions.

To assemble dispenser 100, first, valve assembly 400 is built up, valve 407 is located into flange seal cap 419 and welded to flange 458 of lower valve housing 401. The weld may be the hermetic seal or the compression of the valve flange between the tip and chamber flange. Flange seal 409 is attached to upper valve housing 405 by an ultrasonic bond or alternately an adhesive, chemical bond, or mechanical press fit. Valves 404 and 406 are pressed into upper valve housing 405, and that sub-assembly is then attached to valve housing 401 by a weld, adhesive or mechanical fitment, which can be carried out in discrete steps can happen concurrently, before or after the previous step.

The following assembly steps may be performed in any order, but could be performed after the previously discussed steps to reduce risk of damage to non-plastic parts, where sheath 408 is seated into flange seal 409 in seat 412, this fitment may be a press fit, solvent bond or adhesive bond. Filter 402 is attached by heat bond, press fit or adhesive. Cannula 410 is inserted as a last step to reduce risk of damage to tip 450. Alternatively, cannula 410 is attached to upper valve housing 405 prior to 405 being attached to 401. This allows for an adhesive bond to be used in such a way that adhesive remains in vent compartment 442 and is prevented or eliminated from entering any fluid pathways.

As a separate sub-assembly, O-ring 534 can be assembled to piston 530. Gasket 532 would be positioned on piston 530. This sub-assembly can then be placed into the fluid path chamber subassembly of FIG. 3. Spring 525 may be added and held captive on post 527.

All remaining assembly steps would start with housing 101a being placed in a nest/fixture and the previously described sub-assemblies being located into receiving features in the housing. Ball 580 would be placed in cavity 581. Additionally, carriage member 103 would be placed into the housing and rotationally oriented to its start position. Edge 103c establishes rotational orientation of the carriage in the housing. Button 105 would be placed into the housing, spring 528 would be place with button 105 and held captive on post 529. Feature 284 of cap tether 280 would be placed into its slot in the housing. Housing 101b would then be place over housing 101a, which together hold all of the inserted elements. The housings halves could be snap fit together, press fit, glued, screwed or welded, or some combination. Cap 283 would then be placed over flange seal cap 419.

In one aspect, device 300 is designed for dedicated use with a single medicament container 99 and/or whereas forcing release of either the carriage member 303 from the carriage receiving member 310 or the medicament container 99 from the carriage member 303 would cause damage, including non-operability, of the device. Device 300 and one medicament container 99 can be collectively provided as a kit. Device 300 and two or more medicament containers 99 can be collectively provided as a kit.

The assembly of devices 100, 200, and 300 are similar and can be automated or performed by hand, performed aseptically or subsequently followed by sterilization. For example, assembly 400a is similar to 400. Thus, starting with subassembly 400a button 305 is added, followed by slider 307 and piston 627 with O ring 534 applied. Orifice 327a allows for assembly of piston 627 after slider 307 and button 305 are assembled. Spring 525 is positioned in pocket 627a. All these parts being assembled in a common direction allows for high speed automated assembly without need for reorientation of the assembly. Spring 305a is placed on post 505. Spring 305b is placed in pocket 505b. This now built up sub assembly can be placed into housing 301b. Gravity stop member 330 is placed into receiving feature 331b. Carriage member 103 can then be placed into the housing and rotationally oriented to its start position. Edge 103c establishes rotational orientation of the carriage 303 in the housing 301. Housing 301a is placed over housing 301b thus locating all of the sub-assemblies and components. The housings can be snapped, glued, welded, press fit, screwed together or by some combination thereof. Alternative assembly methods can be used.

To pierce, penetrate or otherwise insert a cannula through the small opening of the container 99, a force of between about 20-30 pounds (about 9.072-13.61 kilograms) or more may be required. In one aspect, the male threads 103a and the female threads 110a of the container manipulation system are configured to provide a reduction of the force required. In one aspect, the devices 100, 200, 300 herein provide a reduction of force of about 2-10 times is provided to pierce, penetrate or otherwise insert a cannula through the small opening of the container 99 requiring about 2-10 lbs (about 0.9072-4.536 kilogram). This reduction in force required is provided by, for example, the pitch and/or thickness of the male/female threads, and/or the sharpness of the piercing distal end 450 of cannula 410, among other parameters.

The components of the devices disclosed may be injection molded, 3D printed, or machined. Anti-microbial material, chemical coatings, and/or lubricants can be added to the molded components of the device to impart microbial control and/or other function, for example, the flow path components, that come into contact the liquid of the medicament container during use, can be coated with anti-absorbing and/or repellant coatings. The velocity of the piston during expulsion of liquid from the device can be adjusted in the present devices such that large molecule or biomolecules, or otherwise sensitive medicinal formulations are not subject to excessive shear or other stress. Furthermore, while certain embodiments of the present disclosure have been illustrated with reference to specific combinations of elements, various other combinations may also be provided without departing from the teachings of the present disclosure. While the devices disclosed provide for application to use for ophthalmological applications, other applications, for example, to mucus membranes, mouth, nose, or ear are envisioned.

Because of the disclosed function of the devices disclosed, a much greater proportion of treatment liquid medicament will actually make contact with the eye, leading to less waste, reduced risk of systemic absorption, less flooding of the eye, and a reduced risk of non-administration because of blinking.

Devices according to the present disclosure may also include a number of additional safety features which are already well established in dosing devices of various kinds, for example, the medicament container 99 will of course have a finite capacity, and a dose recorder may be included to provide an indication of the number of doses remaining or delivered.

Thus, the present disclosure should not be construed as being limited to the particular exemplary embodiments described herein and illustrated in the Figures, but may also encompass combinations of elements of the various illustrated embodiments and aspects thereof.

1. A dispensing device, comprising:
a housing;
a fluid flow system in the housing comprising:
a valve assembly comprising a fluid inlet, a vent inlet and a fluid outlet,
a cannula in fluidic communication with the vent inlet,
a sheath surrounding the cannula and providing a fluid path between the cannula and the sheath, the sheath in fluid communication with the fluid inlet and the fluid outlet,
a piston-cylinder assembly configured to draw a liquid from a container through the fluid path and dispense a liquid;
an actuator assembly providing stored energy and release of the stored energy, the actuator assembly coupled to the piston-cylinder assembly to actuate the piston-cylinder assembly;
a container manipulation system configured to align and present a container with the cannula and the sheath comprising:
a carriage member supporting the container movable relative to the housing between a first position and a second position wherein in the first position the container is not engaged by the cannula and sheath and in the second position the container is engaged by the cannula and sheath such that the container is in fluid communication with the fluid path.

2. The dispensing device of claim 1 wherein the carriage member is threadably engaged with the housing.

3. The dispensing device of claim 2 wherein the carriage member comprises anti-reversing tabs that prevent the carriage member from being threadably disengaged from the housing.

4. The dispensing device of claim 2 wherein the carriage member comprises a deflecting member movable between a first position in which the carriage member is prevented from moving relative to the housing between the first position and the second position and a second position in which the carriage member is free to move relative to the housing between the first position and the second position.

5. The dispensing device of claim 4 wherein the deflecting member is moved between the first position and the second position by the container.

6. A dispensing system comprising:
a housing;
a fluid flow system in the housing comprising:
a valve assembly comprising a fluid inlet, a vent inlet and a fluid outlet,
a cannula in fluidic communication with the vent inlet,
a sheath surrounding the cannula and providing a fluid path between the cannula and the sheath, the sheath in fluid communication with the fluid inlet and the fluid outlet,
a piston-cylinder assembly comprising a cylinder and a piston movable within the cylinder configured to draw a liquid from a container through the fluid path and dispense the liquid via a dispensing valve;
an actuator assembly for actuating the piston-cylinder assembly comprising:
a first actuator and a second actuator, wherein movement of the first actuator between a first position and a second position moves the piston in the cylinder to draw a liquid from a container through the fluid path, stores a biasing energy and allows the second actuator to move from a first position to a second position, and movement of the second actuator from the second positon to the first position releases the stored biasing energy to move the piston in the cylinder to dispense liquid via the dispensing valve.

7. The dispensing device of claim 6 wherein the first actuator and the second actuator move in a linear path.

8. The dispensing device of claim 6 wherein movement of the first actuator between a first position and a second position engages a cam surface on the first actuator with a cam following surface operably connected to the piston to move the piston.

9. The dispensing device of claim 8 wherein movement of the piston compresses a spring to generate the biasing energy.

10. The dispensing device of claim 8 wherein the second actuator is biased from the first positon to the second position and the piston engages a surface on the second actuator to prevent movement of the second actuator from the first position to the second position when the first actuator is in the first position.

11. The dispensing device of claim 10 wherein when the first actuator is in the second position the piston is disengaged from the surface and the second actuator is allowed to move from the first position to the second positon.

12. The dispensing device of claim 10 wherein a plurality of teeth on the piston engage a plurality of teeth on the surface.

13. The dispensing device of claim 10 wherein the second actuator is biased from the first positon to the second position by a spring.

14. The dispensing device of claim 6 comprising a gravity actuated stop that prevents movement of the first actuator when the dispensing device is misoriented.

15. A dispensing device, comprising:
a piston-cylinder assembly comprising a cylinder and a piston movable within the cylinder,
a fluidic system fluidically coupled to the piston-cylinder assembly, the fluidic system comprising a valve assembly, the valve assembly comprising:
a cannula having a piercing distal end; a proximal end separated by a length; and at least one side port in proximity to the piercing distal end; and the at least one side port fluidically coupled to a vent;
a one-way valve fluidically coupled to the vent;
a sheath surrounding the length of cannula, the sheath having a distal end; a proximal end; at least one side opening; and a fluid path between the at least one side opening and a portion of the length of the cannula;
a one-way valve fluidically coupled to the flow path;
an outlet in fluid communication with the cylinder and the flow path;
the piston configured to draw an amount of liquid through the valve assembly into the cylinder, and to expel the amount of liquid; and
an actuator assembly operatively coupled to the piston comprising: a first actuator and a second actuator, wherein movement of the first actuator between a first position and a second position moves the piston in the cylinder to draw a liquid from a container through the fluid path, stores a biasing energy and allows the second actuator to move from a first position to a second position, and movement of the second actuator from the second positon to the first position releases the stored biasing energy to move the piston in the cylinder to dispense liquid via the dispensing valve.

16. The dispensing device of claim 15 further comprising a container manipulation system configured to align and present a container with the cannula and the sheath comprising a carriage member supporting the container movable relative to the housing between a first position and a second position wherein in the first position the container is not engaged by the cannula and sheath and in the second position the container is engaged by the cannula and sheath such that the container is in fluid communication with the fluid path.

17. The dispensing device of claim 15 wherein movement of the first actuator between a first position and a second position engages a cam surface on the first actuator with a cam following surface operably connected to the piston to move the piston.

18. The dispensing device of claim 17 wherein the second actuator is biased from the first positon to the second position and the piston engages a surface on the second actuator to prevent movement of the second actuator from the first position to the second position when the first actuator is in the first position.

19. The dispensing device of claim 18 wherein when the first actuator is in the second position the piston is disengaged from the surface and the second actuator is allowed to move from the first position to the second positon.

20. The dispensing device of claim 18 wherein a plurality of teeth on the piston engage a plurality of teeth on the surface.
